(19) United States
(12) Patent Application Publication
(10) Pub. No.: US 2002/0094335 A1
(43) Pub. Date: Jul. 18, 2002

(54) Vaccine for the prevention and treatment of alzheimer's and amyloid related diseases

(76) Inventors: Robert Chalifour, Ile Bizard (CA); Lise Hebert, Brossard (CA); Xianqi Kong, Dollard-des-Oremaux (CA); Francine Gervais, Ile Bizard (CA)

Correspondence Address: Ivor R. Elrifi, Ph.D., MINTZ, LEVIN, COHN, FERRIS,, GLOVSKY and POPEO, P.C., One Financial Center, Boston, MA, 02111, US

(21) Appl. No.: 09/867,847

(22) Filed: May 29, 2001

Related U.S. Application Data
(63) Continuation-in-part of application No. 09/724,842, filed on Nov. 28, 2000.
(60) Provisional application No. 60/168,594, filed on Nov. 29, 1999.

(51) Int. Cl. A61K 39/00

(52) U.S. Cl. 424/185.1

(57) ABSTRACT

The present invention relates to a stereochemically based “non-self” antigen vaccine for the prevention and/or treatment of Alzheimer's and other amyloid related diseases. The present invention provides a vaccine for the prevention and treatment of Alzheimer's and other amyloid related diseases, which overcomes the drawbacks associated with using naturally occurring peptides, proteins or immunogens.

### RELATED APPLICATIONS

This application claims the benefit of priority under 35 U.S.C. 119(e) to copending U.S. Provisional Application No. 60/168,594, filed on Nov. 29, 1999, the entire contents of which are incorporated herein by reference.

### BACKGROUND OF THE INVENTION

The present invention relates to a new stereochemically based “non-self” antigen vaccine for the prevention and/or treatment of Alzheimer's and other amyloid related diseases.

Amyloidosis refers to a pathological condition characterized by the presence of amyloid fibers. Amyloid is a generic term referring to a group of diverse but specific protein deposits (intracellular and/or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common x-ray diffraction and infrared spectra.

Amyloid-related diseases can either be restricted to one organ or spread to several organs. The first instance is referred to as “localized amyloidosis” while the second is referred to as “systemic amyloidosis”.

Some amyloidotic diseases can be idiopathic, but most of these diseases appear as a complication of a previously existing disorder. For example, primary amyloidosis can appear without any other pathology or can follow plasma cell dyscrasia or multiple myeloma. Secondary amyloidosis is usually seen associated with chronic infection (such as tuberculosis) or chronic inflammation (such as rheumatoid arthritis). A familial form of secondary amyloidosis is also seen in Familial Mediterranean Fever (FMF). This familial type of amyloidosis, as one of the other types of familial amyloidosis, is genetically inherited and is found in specific population groups. In these two types of amyloidosis, deposits are found in several organs and are thus considered systemic amyloid diseases. Another type of systemic amyloidosis is found in long-term hemodialysis patients. In each of these cases, a different amyloidogenic protein is involved in amyloid deposition.

“Localized amyloidoses” are those that tend to involve a single organ system. Different amyloids are also characterized by the type of protein present in the deposit. For example, neurodegenerative diseases such as scrapie, bovine spongiform encephalitis, Creutzfeldt-Jakob disease and the like are characterized by the appearance and accumulation of a protease-resistant form of a prion protein (referred to as AScr or PrP-27) in the central nervous system. Similarly, Alzheimer's disease, another neurodegenerative disorder, is characterized by neuritic plaques and neurofibrillary tangles. In this case, the plaque and blood vessel amyloid is formed by the deposition of fibrillar Aβ amyloid protein. Other diseases such as adult-onset diabetes (Type II diabetes) are characterized by the localized accumulation of amyloid in the pancreas.

Once these amyloids have formed, there is no known, widely accepted therapy or treatment which significantly dissolves the deposits in situ.

Each amyloidogenic protein has the ability to organize into β-sheets and to form insoluble fibrils which get deposited extracellularly or intracellularly. Each amyloidogenic protein, although different in amino acid sequence, has the same property of forming fibrils and binding to other elements such as proteoglycan, amyloid P and complement component. Moreover, each amyloidogenic protein has amino acid sequences which, although different, will show similarities such as regions with the ability to bind to the glycosaminoglycan (GAG) portion of proteoglycan (referred to as the GAG binding site) as well as other regions which will promote β-sheet formation.

In specific cases, amyloidotic fibrils, once deposited, can become toxic to the surrounding cells. As per example, the Aβ fibrils organized as senile plaques have been shown to be associated with dead neuronal cells and microgliosis in patients with Alzheimer's disease. When tested in vitro, Aβ peptide was shown to be capable of triggering an activation process of microglia (brain macrophages), which would explain the presence of microgliosis and brain inflammation found in the brain of patients with Alzheimer's disease.

In another type of amyloidosis seen in patients with Type II diabetes, the amyloidogenic protein IAPP has been shown to induce β-islet cell toxicity in vitro. Hence, appearance of IAPP fibrils in the pancreas of Type II diabetic patients could contribute to the loss of the β islet cells (Langerhans) and organ dysfunction.

People suffering from Alzheimer's disease develop a progressive dementia in adulthood, accompanied by three main structural changes in the brain: diffuse loss of neurons in multiple parts of the brain; accumulation of intracellular protein deposits termed neurofibrillary tangles; and accumulation of extracellular protein deposits termed amyloid or senile plaques, surrounded by misshapen nerve terminals (dystrophic neurites). A main constituent of these amyloid plaques is the amyloid-β peptide (Aβ), a 40-42 amino-acid protein that is produced through cleavage of the β-amyloid precursor protein (APP). Although symptomatic treatments exist for Alzheimer's disease, this disease cannot be prevented nor cured at this time.

The use of a vaccine to treat Alzheimer's disease is possible in principle (Schenk, D. et al., (1999) Nature 400, 173-177). Schenk et al. show that, in a transgenic mouse model of brain amyloidosis (as seen in Alzheimer's disease), immunization with Aβ peptide inhibits the formation of amyloid plaques and the associated dystrophic neurites. In that study, a vaccine using the human aggregated all-L peptide as immunogen prevented the formation of β-amyloid plaque, astrogliosis and neuritic dystrophy in vaccinated transgenic mice.

However, it is apparent that there are a number of drawbacks to using an endogenous protein as a vaccine (or a protein naturally present in the animal being vaccinated). Some of these drawbacks include:

Possible development of autoimmune disease due to the generation of antibodies against “self” protein.

Difficulty in eliciting an immune response due to the failure of the host immune system to recognize “self” antigens.

Possible development of an acute inflammatory response.

### SUMMARY OF THE INVENTION

The present invention relates to a stereochemically based “non-self” antigen vaccine for the prevention and/or treatment of Alzheimer's and other amyloid related diseases. One aim of the present invention is to provide a vaccine for the prevention and treatment of Alzheimer's and other amyloid related diseases, which overcomes the drawbacks associated with using naturally occurring peptides, proteins or immunogens, such as a fibril protein (e.g., Beta Amyloid).

The term “fibril and/or amyloid peptide” as used herein, encompasses both monomeric and oligomeric β-amyloid or any fibril protein as discussed herein, as well as any other structural variants that may occur naturally, are synthetically constructed or correspond to a known fibril protein. Specifically, a fibril and/or amyloid peptide consists of at least 3 amino acids from an fibril peptide, such as amyloid or any structural variant thereof. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “amyloid related diseases” includes diseases associated with the accumulation of amyloid which can either be restricted to one organ, “localized amyloidosis”, or spread to several organs, “systemic amyloidosis”. Secondary amyloidosis may be associated with chronic infection (such as tuberculosis) or chronic inflammation (such as rheumatoid arthritis), including a familial form of secondary amyloidosis which is also seen in Familial Mediterranean Fever (FMF) and another type of systemic amyloidosis found in long-term hemodialysis patients. Localized forms of amyloidosis include, without limitation, diabetes type II and any related disorders thereof, neurodegenerative diseases such as scrapie, bovine spongiform encephalitis, Creutzfeldt-Jakob disease, Alzheimer's disease, Cerebral Amyloid Angiopathy, and prion protein related disorders.

The vaccines of the present invention may be used in the prevention and/or treatment of amyloid related diseases, and in the manufacture of medicaments for preventing and/or treating amyloid-related diseases. A fibril peptide or protein can be derived from a fibril precursor protein known to be associated with certain forms of amyloid diseases, as described herein. Such precursor proteins include, but are not limited to, Serum Amyloid A protein (ApoSSA), immunoglobulin light chain, immunoglobulin heavy chain, ApoA1, transthyretin, lysozyme, fibrinogen α chain, gelsolin, cystatin C, Amyloid beta protein precursor (β-APP), Beta₂ microglobulin, prion precursor protein (PrP), atrial natriuretic factor, keratin, islet amyloid polypeptide, a peptide hormone, and synuclein. Such precursors also include mutant proteins, protein fragments and proteolytic peptides of such precursors. In a preferred embodiment, the peptide is effective to induce an immune response directed against a epitope formed by a fibril protein or peptide, with respect to a fibril precursor protein. That is, as described in more detail herein, many fibril-forming peptides or proteins are fragments of such precursor proteins, such as those listed above. When such fragments are formed, such as by proteolytic cleavage, epitopes may be revealed that are not present on the precursor and are therefore not immunologically available to the immune system when the fragment is a part of the precursor protein.

In another embodiment, the peptide is effective to induce an immune response directed against a epitope formed by a fibril or amyloid protein or peptide. The terms “Aβ,” “Aβ peptide” and “Amyloidβ” peptide are synonymous, and refer to one or more peptide compositions of about 38-43 amino acids derived from Beta Amyloid Precursor Protein (β-APP), as described herein. Disaggregated Aβ means soluble, monomeric and oligomeric peptide units of Aβ. One method to prepare monomeric Aβ is to dissolve lyophilized peptide in neat DMSO with sonication. The resulting solution is centrifuged to remove any insoluble particulates. Aggregated Aβ is a mixture of oligomers in which the monomeric units are held together by noncovalent bonds. Furthermore, APP⁶⁹⁵, APP⁷⁵¹, and APP⁷⁷⁰ refer, respectively, to the 695, 751, and 770 amino acid residue long polypeptides encoded by the human APP gene. See Kang et al., Nature 325, 773 (1987); Ponte et al., Nature 331, 525 (1988); and Kitaguchi et al., Nature 331, 530 (1988). Amino acids within the human amyloid precursor protein (APP) are assigned numbers according to the sequence of the APP77O isoform. Terms such as Aβ39, Aβ40, Aβ41, Aβ42 and Aβ43 refer to an Aβ peptide containing amino acid residues 1-39, 1-40, 1-41, 1-42 and 1-43. Preferably, Aβ peptide contains amino acid residues 13-21. More preferably, the Aβ peptide contains amino acid residues 10-21.

Accordingly, in one embodiment of the present invention, a vaccine is provided which is produced using a “non-self” peptide or protein synthesized from the unnatural D-configuration amino acids, to avoid the drawbacks of using “self” proteins. The peptides need not be aggregated to be operative or immunogenic as opposed to the prior art vaccines. Thus, an “immunogenic peptide,” “immunogenic peptide” or “immunogen” or “antigen” is a molecule that is capable of inducing an immunological response against itself upon administration to a patient, either in conjunction with, or in the absence of, an adjuvant. Such molecules include, for example, amyloid fibril peptides or fragments thereof conjugated to a carrier protein, such as keyhole limpet hemocyanin, Cd3 or tetanus toxin.

The term “immunological” or “immune” or “immunogenic” response refers to the development of a humoral (antibody mediated) and/or a cellular (mediated by antigen-specific T cells or their secretion products) response directed against an antigen in a vertebrate individual. Such a response can be an active response induced by administration of immunogen or a passive response induced by administration of antibody or primed T-cells or B cells which can act as antigen presenting cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by standard proliferation assays (CD4⁺ T cells) or CTL (cytotoxic T lymphocyte) assays known in the art. The relative contributions of humoral and cellular responses to the protective or therapeutic effect of an immunogen can be distinguished by separately isolating immunoglobulin (IgG) and T-cell fractions from an immunized syngeneic animal and measuring protective or therapeutic effect in a second subject.

The terms “polynucleotide” and “nucleic acid,” as used interchangeably herein refer to a polymeric molecule having a backbone that supports bases capable of hydrogen bonding to typical polynucleotides, where the polymer backbone presents the bases in a manner to permit such hydrogen bonding in a sequence specific fashion between the polymeric molecule and a typical polynucleotide (e.g., single-stranded DNA). Such bases are typically inosine, adenosine, guanosine, cytosine, uracil and thymidine. Polymeric molecules include double and single stranded RNA and DNA, and backbone modifications thereof, for example, methylphosphonate linkages. The term “polypeptide” as used herein refers to a compound made up of a single chain of amino acid residues linked by peptide bonds. The term “protein” may be synonymous with the term “polypeptide” or may refer to a complex of two or more polypeptides. The term “peptide” also refers to a compound composed of amino acid residues linked by peptide bonds. Generally peptides are composed of 100 or fewer amino acids, while polypeptides or proteins have more than 100 amino acids. As used herein, the term “protein fragment” may also be read to mean a peptide.

Except as otherwise expressly defined herein, the abbreviations used herein for designating the amino acids and the protective groups are based on recommendations of the IUPAC-IUB Commission on Biochemical Nomenclature (Biochemistry, 1972, 11:1726-1732).

In another embodiment, there is provided a method for preventing and/or treating an amyloid-related disease in a subject, which features administering to the subject an antigenic amount of an all-D peptide which elicits production of antibodies against the all-D peptide, and elicit an immune response by the subject, therefore preventing fibrillogenesis, associated cellular toxicity and neurodegeneration, wherein the antibodies interact with at least one region of a fibril or amyloid protein, a fibril protein or another non-amyloid protein which induces amyloidosis. A “fibril peptide” or “fibril protein” refers to a monomeric and oligomeric or aggregated form of a protein or peptide that forms fibrils present in amyloid plaques. Examples of such peptides and proteins are provided herein. “Nonamyloid protein” containing formulations include, but are not limited to: compositions that produce immune responses against gelsolin fragments for treatment of hereditary systemic amyloidosis, mutant lysozyme protein (Alys), for treatment of a hereditary neuropathy, mutant alpha chain of fibrinogen (AfibA) for a non-neuropathic form of amyloidosis manifest as renal disease, mutant cystatin C (Acys) for treatment of a form of hereditary cerebral angiopathy reported in Iceland. In addition, certain hereditary forms of prion disease (e.g., Creutzfeldt-Jacob disease (CJD), Gerstmann-Sträussler-Scheinker syndrome (GSS), and fatal familial insomnia (FFI)) are characterized by a mutant isoform of prion protein, PrP^{Sc}.This protein can be used in therapeutic compositions for treatment and prevention of deposition of PrP plaques, in accordance with the present invention. These vaccines may be used in the prevention and/or treatment of amyloid related diseases, and in the manufacture of medicaments for preventing and/or treating amyloid-related diseases.

In a further embodiment of the invention, a vaccine for preventing and/or treating an amyloid-related disease in a subject comprises at least one antibody which interacts with either amyloid proteins or fibril proteins to prevent fibrillogenesis, wherein the antibodies are raised against an antigenic amount of an all-D peptide of either protein, e.g., β sheet region and GAG-binding site region, Aβ(1-42, all-D) and macrophage adherence region Aβ(10-16), immunogenic fragments thereof, protein conjugates thereof, immunogenic derivative peptides thereof, immunogenic peptides thereof, and immunogenic peptidomimetics thereof, or a peptide which has a substantial identity to any of the above peptides.

The following terms are used to describe the sequence relationships between two or more polynucleotides: “substantial identity”, “comparison window”, “sequence identity”, “percentage of sequence identity”, and “reference sequence.” A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “antibody” or “immunoglobulin” is used to include intact antibodies and binding fragments thereof. Typically, fragments compete with the intact antibody from which they were derived for specific binding to an antigen fragment including separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Specific binding between two entities means an affinity of at least 10⁶ M⁻¹, 10⁷ M⁻¹, 10⁸ M⁻¹, 10⁹M⁻¹, or 10¹⁰M⁻¹. Affinities greater than 10⁸ M⁻¹ are preferred.

In another embodiment, there is provided a method for preventing and/or treating an amyloid-related disease in a subject which comprises administering a pharmaceutical composition comprising a combination of at least two antibodies that bind to two or more portions of a fibril protein and/or a nonamyloid protein.

A “pharmaceutical composition” refers to a chemical or biological composition suitable for administration to a mammalian individual. Such compositions may be specifically formulated for administration via one or more of a number of routes, including but not limited to, oral, parenteral, intravenous, intraarterial, subcutaneous, intranasal, sublingual, intraspinal, intracerebroventricular, and the like. A “pharmaceutical excipient” or a “pharmaceutically acceptable excipient” is a carrier, usually a liquid, in which an active therapeutic peptide is formulated. The excipient generally does not provide any pharmacological activity to the formulation, though it may provide chemical and/or biological stability, release characteristics, and the like. Exemplary formulations can be found, for example, in Remington's Pharmaceutical Sciences, 19^{th} Ed., Grennaro, A., Ed., 1995.

Still in a further embodiment, there is provided a method for preventing and/or treating an amyloid-related disease in a subject, which comprises administering to the subject an antigenic amount of an all-D peptide which interacts with at least one region of a fibril or amyloid protein, e.g., β sheet region and GAG-binding site region, Aβ(1-42) and macrophage adherence region Aβ(10-16), immunogenic fragments thereof, protein conjugates thereof, immunogenic derivative peptides thereof, immunogenic peptides thereof, and immunogenic peptidomimetics thereof, wherein the compound elicits an immune response by the subject and therefore prevents fibrillogenesis.

According to another related aspect, the invention includes a method of preventing or treating a disorder characterized by amyloid deposition in a mammalian subject. In accordance with this aspect of the invention, the subject is given a dosage of an peptide effective to produce an immune response against an fibril and/or amyloid peptide characteristic of the amyloid disorder from which the subject suffers. Essentially, the methods include administering pharmaceutical compositions containing immunogenic fibril and/or amyloid peptides specific to the disorder, such as those described above. Such methods are further characterized by their effectiveness in inducing immunogenic responses in the subject. According to a preferred embodiment, the method is effective to produce an immunological response that is characterized by a serum titer of at least 1:1000 with respect to the fibril and/or amyloid peptide against which the immunogenic peptide is directed. In yet a further preferred embodiment, the serum titer is at least 1:5000 with respect to the fibril component. According to a related embodiment, the immune response is characterized by a serum amount of immunoreactivity corresponding to greater than about four times higher than a serum level of immunoreactivity measured in a pre-treatment control serum sample. This latter characterization is particularly appropriate when serum immunoreactivity is measured by ELISA techniques, but can apply to any relative or absolute measurement of serum immunoreactivity. According to a preferred embodiment, the immunoreactivity is measured at a serum dilution of about 1:100.

According to a still further related aspect, the invention includes a method of determining the prognosis of a patient undergoing treatment for an amyloid disorder. Here, patient serum amount of immunoreactivity against an fibril and/or amyloid peptide characteristic of the selected disorder is measured, and a patient serum amount of immunoreactivity of at least four times a baseline control level of serum immunoreactivity is indicative of a prognosis of improved status with respect to the particular amyloid disorder. According to preferred embodiments, the amount of immunoreactivity against the selected fibril and/or amyloid peptide present in the patient serum is characterized by a serum titer of at least about 1:1000, or at least 1:5000, with respect to the fibril and/or amyloid peptide.

According to a still related aspect, the invention also includes so-called “passive immunization” methods and pharmaceutical compositions for preventing or treating amyloid diseases. According to this aspect of the invention, patients are given an effective dosage of an antibody that specifically binds to a selected fibril and/or amyloid peptide, preferably a fibril component present in amyloid deposits characteristic of the disease to be treated. In general, such antibodies are selected for their abilities to specifically bind the various proteins, peptides, and components described with respect to the pharmaceutical compositions and methods described in the preceding paragraphs of this section. According to a related embodiment, such methods and compositions may include combinations of antibodies that bind at least two amyloid fibril components. In general, pharmaceutical compositions are administered to provide a serum amount of immunoreactivity against the target fibril and/or amyloid peptide that is at least about four times higher than a serum level of immunoreactivity against the component measured in a control serum sample. The antibodies may also be administered with a carrier, as described herein. In general, in accordance with this aspect of the invention, such antibodies, will be administered (or formulated for administration) pentoneally, orally, intranasally, subcutaneously, intramuscularly, topically or intravenously, but can be administered or formulated for administration by any pharmaceutically effective route (i.e., effective to produce the indicated therapeutic levels, as set forth above and herein).

The antibody can also be a monoclonal antibody.

The term “epitope” or “antigenic determinant” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen. 1-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J. Inf Dis. 170, 1110-19 (1994)), by antigen-dependent killing (cytotoxic T lymphocyte assay, Tigges et al., J. Immunol. 156, 3901-3910) or by cytokine secretion.

As used herein, the term “compound” refers to a peptide of the present invention or a pharmaceutically acceptable composition containing a peptide according to the present invention. In a preferred embodiment of the present invention, the compound is a compound of Formula I:

R′—(P)—R″ (I),

wherein

P is an all-D peptide of a fibril or amyloid protein, e.g., β sheet region, GAG-binding site region, Aβ(1-42, all-D), and macrophage adherence region (10-16, all-D) immunogenic fragments thereof, immunogenic derivatives thereof, protein conjugates thereof, immunogenic peptides thereof, and immunogenic peptidomimetics thereof;

R′ is an N-terminal substituent, e.g.:

hydrogen;

lower alkyl groups, e.g., acyclic or cyclic having 1 to 8 carbon atoms, without or with functional groups, e.g., carboxylate, sulfonate and phosphonate;

aromatic groups;

heterocyclic groups; and

acyl groups, e.g., alkylcarbonyl, arylcarbonyl, sulfonyl and phosphonyl groups; and

R″ is a C-terminal substituent, e.g., hydroxy, alkoxy, aryloxy, unsubstituted or substituted amino groups.

In an embodiment, R′ and R″ are identical or different, wherein alkyl or aryl group of R′ and R″ are further substituted with functionalities such as halide (e.g., F, Cl, Br, and I), hydroxyl, alkoxyl, aryloxyl, hydroxycarbonyl, alkoxylcarbonyl, aryloxycarbonyl, carbamyl, unsubstituted or substituted amino, sulfo or alkyloxysulfonyl, phosphono or alkoxyphosphonyl groups.

When the compound has an acid functional group, it can be in the form of a pharmaceutically acceptable salt or ester. When the compound has a basic functional group, it can be in the form of a pharmaceutically acceptable salt.

In one embodiment, P is a peptide capable of interacting with at least one region of a fibril or amyloid protein.

In a preferred embodiment of the present invention, the subject is a human being.

In yet another embodiment of the present invention, the amyloid related disease may be Alzheimer's disease.

In still another embodiment of the present invention, the amyloid related disease may be Cerebral Amyloid Angiopathy (CAA).

In another embodiment of the present invention, there is provided a method for preventing and/or treating of an amyloid related disease in a subject, comprising administering to the subject an antigenic amount of a compound of Formula I:

R′—(P)—R″ (I),

wherein

P is an all-D peptide of a fibril or amyloid protein, e.g., β sheet region, GAG-binding site region, Aβ(1-42, all-D), and macrophage adherence region (10-16, all-D) immunogenic fragments thereof, immunogenic derivatives thereof, protein conjugates thereof, immunogenic peptides thereof, and immunogenic peptidomimetics thereof;

R′ is an N-terminal substituent selected from the group consisting of:

hydrogen;

lower alkyl groups, e.g., acyclic or cyclic having 1 to 8 carbon atoms, without or with functional groups, e.g., carboxylate, sulfonate and phosphonate;

aromatic groups;

heterocyclic groups; and

acyl groups, e.g., alkylcarbonyl, arylcarbonyl, sulfonyl and phosphonyl groups; and

R″ is a C-terminal substituent, e.g., hydroxy, alkoxy, aryloxy, unsubstituted or substituted amino groups.

In accordance with this method, the compound elicits an immune response by the subject, preventing fibrillogenesis and neurodegeneration.

In accordance with a preferred embodiment of the present invention, there is provided a vaccine for preventing and/or treating an amyloid-related disease in a subject, comprising an antigenic amount of an all-D peptide which interacts with at least one region of a fibril or amyloid protein, e.g., β sheet region, GAG-binding site region, Aβ(1-42, all-D) peptide, and macrophage adherence region (10-16, all-D) immunogenic fragments thereof, protein conjugates thereof, immunogenic derivative peptides thereof, immunogenic peptides thereof, and immunogenic peptidomimetics thereof, wherein the compound elicits an immune response by the subject and prevents fibrillogenesis.

In another embodiment, some methods entail administering an effective dosage of at least one antibody that specifically binds to a fibril protein. Preferably, the present invention embodies administering at least one antibody that specifically binds to an epitope within residues 1-42 of Aβ. In some methods, the antibody specifically binds to an epitope within residues 15-20 of Aβ. In some methods, the antibody specifically binds to an epitope within residues 13-21 of Aβ. In some methods, the antibody specifically binds to an epitope within residues 10-21 of Aβ. In some methods, the antibody specifically binds to an epitope within residues 10-16 of Aβ. In some methods, the antibody specifically binds to an epitope within residues 25-35 of Aβ. In some methods, the antibody binds to an epitope comprising a free N-terminal residue of Aβ.

The methods can be used on both asymptomatic patients and those currently showing symptoms of disease. The antibody used in such methods can be a human, humanized, chimeric or nonhuman antibody and can be monoclonal or polyclonal. In some methods, the antibody is prepared from a human immunized with Aβ peptide, which human can be the patient to be treated with antibody.

In some methods, the antibody is administered with a pharmaceutical carrier as a pharmaceutical composition. In some methods, antibody is administered at a dosage of 0.0001 to 100 mg/kg, preferably, at least 1 mg/kg body weight antibody. In some methods, the antibody is administered in multiple dosages over a prolonged period, for example, of at least six months. In some methods, the antibody is administered as a sustained release composition. The antibody can be administered, for example, intraperitoneally, orally, subcutaneously, intracranially, intramuscularly, topically, intranasally or intravenously.

Other features and advantages of the invention will be apparent from the following detailed description and claims.

### BRIEF DESCRIPTION OF THE DRAWING

FIG. 1 illustrates the targeted sites for the antigenic fragments.

FIG. 2 illustrates the effect of 1 mg/ml of antibodies raised against D and L forms of Aβ(16-21) on fibrillogenesis.

FIG. 3 illustrates the effect of 0.5 mg/ml of antibodies raised against D and L forms of Aβ(16-21) on fibrillogenesis.

FIGS. 4A to 4C illustrate electron micrographs showing the effect of anti-D KLVFFA peptide antibodies (FIG. 4B) and anti-L KLVFFA peptide antibodies (FIG. 4C) with respect to a control (FIG. 4A) on fibrillogenesis.

FIGS. 5A to 5D illustrate the immunohistochemistry of anti-D KLVFFA on aggregated Aβ peptide in brain sections of retrosplenial cortex (FIG. 5A) and parietal cortex (FIG. 5C) and the histochemistry (Thioflavin S assay) of anti-D KLVFFA on aggregated Aβ peptide in the same brain sections of retrospinal cortex (FIG. 5B) and parietal cortex (FIG. 5D).

FIGS. 6A to 6D illustrate the immunohistochemistry of anti-L KLVFFA antibodies on aggregated Aβ peptide in brain sections of parietal cortex (FIG. 6A) and entorhinal cortex (FIG. 6C) and the histochemistry (Thioflavin S assay) of anti-L KLVFFA antibodies on aggregated Aβ peptide in the same brain sections of parietal cortex (FIG. 6B) and entorhinal cortex (FIG. 6D).

FIG. 7 illustrates the response of rabbits to KLH-conjugated all-L and all-D KLVFFA.

### DETAILED DESCRIPTION OF THE INVENTION

Amyloid diseases or amyloidoses include a number of disease states having a wide variety of outward symptoms. These disorders have in common the presence of abnormal extracellular deposits of protein flbrils, known as “amyloid deposits” or “amyloid plaques” that are usually about 10-100 nm in diameter and are localized to specific organs or tissue regions. Such plaques are composed primarily of a naturally occurring soluble protein or peptide. These insoluble deposits are composed of generally lateral aggregates of fibrils that are approximately 10-15 nm in diameter. Amyloid fibrils produce a characteristic apple green birefringence in polarized light, when stained with Congo Red dye.

The peptides or proteins forming the plaque deposits are often produced from a larger precursor protein. More specifically, the pathogenesis of amyloid fibril deposits generally involves proteolytic cleavage of an “abnormal” precursor protein into fragments. These fragments generally aggregate into anti-parallel β-pleated sheets; however, certain undegraded forms of precursor protein have been reported to aggregate and form fibrils in familial amyloid polyneuropathy (variant transthyretin fibrils) and dialysis-related amyloidosis (β₂ microglobulin fibrils) (Tan, et al., 1994, supra).

Specifically, the Aβ(16-21) site is known to play an important role in initiating the harmful process of Aβ peptide amyloidogenesis. It is also known that when these peptides are made from D-amino acids, they retain their ability to interact with the natural all-L-homologous sequence, thereby preventing amyloidogenesis. Other amyloid proteins which may be used in the present invention include, without limitation, the beta sheet region of IAPP (24-29, all-D), β2-microglobulin, amyloid A protein, and prion-related proteins.

The disorders are classified on the basis of the major fibril components forming the plaque deposits, as discussed below.

Amyloid Diseases

The present invention is based on the discovery that amyloid diseases can be treated by administering peptides that serve to stimulate an immune response against a component or components of the various disease-specific amyloid deposits. The sections below serve to exemplify major forms of amyloidosis and are not intended to limit the invention.

AA (Reactive) Amyloidosis

Generally, AA amyloidosis is a manifestation of a number of diseases that provoke a sustained acute phase response. Such diseases include chronic inflammatory disorders, chronic local or systemic microbial infections, and malignant neoplasms.

AA fibrils are generally composed of 8000 dalton fragments (AA peptide or protein) formed by proteolytic cleavage of serum amyloid A protein (apoSSA), a circulating apolipoprotein which is present in HDL complexes and which is synthesized in hepatocytes in response to such cytokines as IL-1, IL-6 and TNF. Deposition can be widespread in the body, with a preference for parenchymal organs. The spleen is usually a deposition site, and the kidneys may also be affected. Deposition is also common in the heart and gastrointestinal tract.

AA amyloid diseases include, but are not limited to inflammatory diseases, such as rheumatoid arthritis, juvenile chronic arthritis, ankylosing spondylitis, psoriasis, psoriatic arthropathy, Reiter's syndrome, Adult Still's disease, Behcet's syndrome, and Crohn's disease. AA deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in AA fibril ainyloid deposits. These include such conditions as Hodgkin's lymphoma, renal carcinoma, carcinomas of gut, lung and urogenital tract, basal cell carcinoma, and hairy cell leukemia.

AL Amyloidoses

AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia.

Fibrils of AL amyloid deposits are composed of monoclonal immunoglobulin light chains or fragments thereof. More specifically, the fragments are derived from the N-terminal region of the light chain (kappa or lambda) and contain all or part of the variable (V_{L}) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, as well as occult dyscrasias. However, it should be noted that almost any tissue, particularly visceral organs such as the heart, may be involved.

Hereditary Systemic Amyloidoses

There are many forms of hereditary systemic amyloidoses. Although they are relatively rare conditions, adult onset of symptoms and their inheritance patterns (usually autosomal dominant) lead to persistence of such disorders in the general population. Generally, the syndromes are attributable to point mutations in the precursor protein leading to production of variant asnyloidogenic peptides or proteins. Table 1 summarizes the fibril composition of exemplary forms of these disorders.

TABLE 1

| Fibril Peptide/Protein | Genetic variant | Clinical Syndrome |
|---|---|---|
| Transthyretin and fragments | Met30, many others | Familial amyloid polyneuropahty |
| (ATTR) | | (FAP), (Mainly peripheral nerves) |
| Transthyretin and fragments | Thr45, Ala60, Ser84, Met111, | Cardiac involvement predominant |
| (ATTR) | Ile122 | without neuropathy |
| N-terminal fragment of | Arg26 | Familial amyloid polyneurophathy |
| Apolipoprotein A1 (apoAI) | | (FAP), (mainly peripheral nerves) |
| N-terminal fragment of | Arg26, Arg50, Arg 60, others | Ostertag-type, non-neuropathic |
| Apoliproprotein A1 (AapoAI) | | (predominantly visceral |
| | | involvement) |
| Lysozyme (Alys) | Thr56, His67 | Ostertag-type, non-neuropathic |
| | | (predominantly visceral |
| | | involvement) |
| Fibrogen ∀ chain fragment | Leu554, Val 526 | Cranial neuropathy with lattic |
| | | corneal dystrophy |
| Gelsolin fragment (Agel) | Asn187, Tyr187 | Cranial neuropathy with lattice |
| | | cornealk dystrophy |
| Cystatin C fragment | Glu68 | Hereditary cerebral hemmorrhage |
| | | (cerebral amyloid angiopathy) - |
| | | Icelandic type |
| β-amyloid protein (aβ) derived from | Gln693 | Hereditary cerebral hemmorrhage |
| Amyloid Precursor Protein (APP) | | (cerebral amyloid angiopathy) - |
| | | Dutch type |
| β-amyloid protein (aβ) derived from | Ile717, Phe717, Gly717 | Familial Alzheimer's Disease |
| Amyloid Precursor Protein (APP) | | |
| β-amyloid protein (aβ) derived from | Asn670, Leu671 | Familial Dementia - probably |
| Amyloid Precursor Protein (APP) | | Alzheimer's Disease |
| Prion Protein (PrP) derived from Prp | Leu102, Val167, Asn78, Lys200 | Familial Creutzfeldt-Jakob disease; |
| precursor protein | | Gerstmann-Sträussler-Scheinker |
| 51-91 insert | | syndrome (hereditary spongiform |
| | | encephalopathies, prion diseases) |
| AA derived from Serum amyloid A | | Familial Mediterranean fever, |
| protein (ApoSSA) | | predominant renal involvement |
| | | (autosomal recessive) |
| AA derived from Serum amyloid A | | Muckle-Well's syndrome, |
| protein (ApoSSA) | | nephropathy, deafness, urticaria, |
| | | limb pain |
| Unknown | | Cardiomyophathy with persistent |
| | | atrial standstill |
| Unknown | | Cutaneous deposits (bullous, |
| | | papular, pustulodermal) |

*Data derived from Tan & Pepys, 1994, supra.

The data provided in Table 1 is exemplary and are not intended to limit the scope of the invention. For example, more than 40 separate point mutations in the transthyretin gene have been described, all of which give rise to clinically similar forms of familial amyloid polyneuropathy.

Transthyretin (TTR) is a 14 kilodalton protein that is also sometimes referred to as prealbumin. It is produced by the liver and choroid plexus, and it functions in transporting thyroid hormones and vitamin A. At least 50 variant forms of the protein, each characterized by a single amino acid change, are responsible for various forms of familial amyloid polyneuropathy. For example, substitution of proline for leucine at position 55 results in a particularly progressive form of neuropathy; substitution of methionine for leucine at position 111 resulted in a severe cardiopathy in Danish patients. Amyloid deposits isolated from heart tissue of patients with systemic amyloidosis have revealed that the deposits are composed of a heterogeneous mixture of TTR and fragments thereof, collectively referred to as ATTR, the full length sequences of which have been characterized. ATTR fibril components can be extracted from such plaques and their structure and sequence determined according to the methods known in the art (e.g., Gustavsson, A., et al., Laboratory Invest. 73: 703-708, 1995; Kametani, F., et al., Biochem. Biophys. Res. Commun. 125: 622-628, 1984; Pras, M., et al., PNAS 80: 539-42, 1983).

Persons having point mutations in the molecule apolipoprotein Al (e.g., Gly→Arg26; Trp 4→Arg50; Leu→4 Arg60) exhibit a form of amyloidosis (“Östertag type”) characterized by deposits of the protein apolipoprotein AI or fragments thereof (AApoAI). These patients have low levels of high density lipoprotein (HDL) and present with a peripheral neuropathy or renal failure.

A mutation in the alpha chain of the enzyme lysozyme (e.g., Ile→Thr56 or Asp→His57) is the basis of another form of Östertag-type non-neuropathic hereditary amyloid reported in English families. Here, fibrils of the mutant lysozyine protein (Alys) are deposited, and patients generally exhibit impaired renal function. This protein, unlike most of the fibril-forming proteins described herein, is usually present in whole (unfragmented) form (Benson, M. D., et al. CIBA Fdn. Symp. 199: 104-131, 1996).

β-amyloid peptide (Aβ) is a 39-43 amino acid peptide derived by proteolysis from a large protein known as Beta Amyloid Precursor protein (βAPP). Mutations in βAPP result in familial forms of Alzheimer's disease, Down's syndrome and/or senile dementia, characterized by cerebral deposition of plaques composed of βP fibrils and other components, which are described in further detail below. Known mutations in APP associated with Alzheimer's disease occur proximate to the cleavage sites of β or gamma-secretase, or within Aβ. For example, position 717 is proximate to the site of gamma-secretase cleavage of APP in its processing to Aβ, and positions 670/671 are proximate to the site of β-secretase cleavage. Mutations at any of these residues may result in Alzheimer's disease, presumably by causing an increase the amount of the 42/43 amino acid form of Aβ generated from APP. The structure and sequence of Aβ peptides of various lengths are well known in the art. Such peptides can be made according to methods known in the art (e.g., Glenner and Wong, Biochem Biophys. Res. Comm. 129: 885-890, 1984; Glenner and Wong, Biochem Biophys. Res. Comm. 122: 113 1-1135, 1984). In addition, various forms of the peptides are commercially available.

Synuclein is a synapse-associated protein that resembles an alipoprotein and is abundant in neuronal cytosol and presynaptic terminals. A peptide fragment derived from alpha-synuclein, termed NAC, is also a component of amyloid plaques of Alzheimer's disease. (Clayton, et al., 1998). This component also serves as a target for immunologically-based treatments of the present invention, as detailed below.

Gelsolin is a calcium binding protein that binds to fragments and actin filaments. Mutations at position 187 (e.g., Asp→Asn; Asp→Tyr) of the protein result in a form of hereditary systemic amyloidosis, usually found in patients from Finland, as well as persons of Dutch or Japanese origin. In afflicted individuals, fibrils formed from gelsolin fragments (Agel), usually consist of amino acids 173-243 (68 kDa carboxyterminal fragment) and are deposited in blood vessels and basement membranes, resulting in corneal dystrophy and cranial neuropathy which progresses to peripheral neuropathy, dystrophic skin changes and deposition in other organs. (Kangas, H., et al. Human Mol. Genet. 5(9): 1237-1243, 1996).

Other mutated proteins, such as mutant alpha chain of fibrinogen (AfibA) and mutant cystatin C (Acys) also form fibrils and produce characteristic hereditary disorders. AfibA fibrils form deposits characteristic of a nonneuropathic hereditary amyloid with renal disease; Acys deposits are characteristic of a hereditary cerebral amyloid angiopathy reported in Iceland. (Isselbacher, Ct at., Harrison's Principles of Internal Medicine, McGraw-Hill, San Francisco, 1995; Benson, et al., supra.). In at least some cases, patients with cerebral amyloid angiopathy (CAA) have been shown to have amyloid fibrils containing a non-mutant form of cystatin C in conjunction with beta protein. (Nagai, A., et al. Molec. Chem. Neuropathol. 33: 63-78, 1998).

Certain forms of prion disease are now considered to be heritable, accounting for up to 15% of cases, which were previously thought to be predominantly infectious in nature. (Baldwin, et al., in Research Advances in Alzheimer's Disease and Related Disorders, John Wiley and Sons, New York, 1995). In such prion disorders, patients develop plaques composed of abnormal isoforms of the normal prion protein (PrP^{Sc}). A predominant mutant isoform, PrP^{Sc}, also referred to as AScr, differs from the normal cellular protein in its resistance to protease degradation, insolubility after detergent extraction, deposition in secondary lysosomes, post-translational synthesis, and high β-pleated sheet content. Genetic linkage has been established for at least five mutations resulting in Creutzfeldt-Jacob disease (CJD), Gerstmann-Sträussler-Scheinker syndrome (GSS), and fatal familial insomnia (FFI). (Baldwin) Methods for extracting fibril peptides from scrapie fibrils, determining sequences and making such peptides are known in the art. (e.g., Beekes, M., et al. J. Gen. Virol. 76: 2567-76, 1995).

For example, one form of GSS has been linked to a PrP mutation at codon 102, while telencephalic GSS segregates with a mutation at codon 117. Mutations at codons 198 and 217 result in a form of GSS in which neuritic plaques characteristic of Alzheimer's disease contain PrP instead of Aβ peptide. Certain forms of familial CJD have been associated with mutations at codons 200 and 210; mutations at codons 129 and 178 have been found in both familial CJD and FFI. (Baldwin, supra).

Senile Systemic Amyloidosis

Amyloid deposition, either systemic or focal, increases with age. For example, fibrils of wild type transthyretin (TTR) are commonly found in the heart tissue of elderly individuals. These may be asymptomatic, clinically silent, or may result in heart failure. Asymptomatic fibrillar focal deposits may also occur in the brain (Aβ), corpora amylacea of the prostate (Aβ₂ microglobulin), joints and seminal vesicles.

Cerebral Amyloidosis

Local deposition of amyloid is common in the brain, particularly in elderly individuals. The most frequent type of amyloid in the brain is composed primarily of Aβ peptide fibrils, resulting in dementia or sporadic (non-hereditary) Alzheimer's disease. In fact, the incidence of sporadic Alzheimer's disease greatly exceeds forms shown to be hereditary. Fibril peptides forming these plaques are very similar to those described above, with reference to hereditary forms of Alzheimer's disease (AD).

Dialysis-related Amyloidosis

Plaques composed of β₂ microglobulin (Aβ₂M) fibrils commonly develop in patients receiving long term hemodialysis or peritoneal dialysis. β₂ microglobulin is a 11.8 kilodalton polypeptide and is the light chain of Class I MHC antigens, which are present on all nucleated cells. Under normal circumstances, it is continuously shed from cell membranes and is normally filtered by the kidney. Failure of clearance, such as in the case of impaired renal function, leads to deposition in the kidney and other sites (primarily in collagen-rich tissues of the joints). Unlike other fibril proteins, Aβ₂M molecules are generally present in unfragmented form in the fibrils. (Benson, supra).

Hormone-derived Amyloidoses

Endocrine organs may harbor amyloid deposits, particularly in aged individuals. Hormone-secreting tumors may also contain hormone-derived amyloid plaques, the fibrils of which are made up of polypeptide hormones such as calcitonin (medullary carcinoma of the thyroid), islet amyloid polypeptide (amylin; occurring in most patients with Type II diabetes), and atrial natriuretic peptide (isolated atrial amyloidosis). Sequences and structures of these proteins are well known in the art.

Miscellaneous Amyloidoses

There are a variety of other forms of amyloid disease that are normally manifest as localized deposits of amyloid. In general, these diseases are probably the result of the localized production and/or lack of catabolism of specific fibril precursors or a predisposition of a particular tissue (such as the joint) for fibril deposition. Examples of such idiopathic deposition include nodular AL amyloid, cutaneous amyloid, endocrine amyloid, and tumor-related amyloid.

Pharmaceutical Compositions

The pharmaceutical compositions of the present invention are directed to vaccines prepared from fibril peptides that have at least 50% of their amino acid residues in the dextro form (D-isomers). Preferably, the vaccines are prepared from all D-Aβ(10-21), D-Aβ(13-21), D-Aβ(25-35), D-Aβ(16-21), D-Aβ(10-16), D-Aβ(1-40), D-Aβ(1-42) or the C-terminal regions of D-Aβ(1-42), is believed to elicit an immune response in the host or in producing antibodies that recognize the naturally occurring target. As used herein, “all-D” includes peptides having at least 50% D-configuration amino acids. Preferably, “all-D” also includes peptides having greater than or equal to 50%; 55%; 60%; 65%; 70%;75%; 80%; 85%; 90%; 95% or 100%

D-configuration Amino Acids

The vaccine according to the present invention is able to prevent the development of brain amyloidosis through two possible scenerios: 1) the effect of anti-Aβ antibodies at the site of amyloid deposition, and 2) the systemic effect of the high circulatory anti-Aβ level on the plasmatic Aβ concentrations.

Specifically, elevated plasma anti-Aβ antibody levels may act systemically by decreasing normal Aβ plasma levels, thereby creating a systemic imbalance in the normal Aβ levels. Such an imbalance could lead to the activation of the mechanism responsible for the clearing in Aβ levels from the brain, in order to re-establish the normal balance between brain and plasma Aβ levels.

Accordingly, this possibility could be exploited by determining the effect of active or passive immunization on plasma and brain Aβ40 levels at different timepoints following such immunization. Aβ-immunization can also exert a systemic protective effect versus the development of brain amyloidosis. The ratio of Aβ levels in plasma and brain should remain constant in immunized transgenic animals, while it should decrease in the control animals. Additionally, B-cell or bone marrow cell transfer from immunized to naive transgenic animals should have the same effect as passive immunization using anti-Aβ antibodies.

Furthermore, the vaccine of the present invention does not present the drawbacks of using “self” proteins and does not need to be aggregated to induce an immune response. For example, the antibodies raised against the all-D-Aβ(16-21) peptide can be expected to recognize the all-L-Aβ(16-21) peptide sequence. Pharmaceutical compositions of the present invention may include, in addition to the immunogenic peptide(s), an effective amount of an adjuvant and/or an excipient. Pharmaceutically effective and useful adjuvants and excipients are well known in the art, and are described in more detail below.

According to the present invention, compositions capable of eliciting or providing an immune response directed to certain components of amyloid plaques are effective to treat or prevent development of amyloid diseases. In particular, according to the invention provided herein, it is possible to prevent progression of, ameliorate the symptoms of, and/or reduce amyloid plaque burden in afflicted individuals, when an immunostimulatory dose of an anti-amyloid peptide, or corresponding anti-amyloid immune repeptide, is administered to the patient. This section describes exemplary anti-amyloid peptides that produce active, as well as passive, immune responses to amyloid plaques and provides exemplary data showing the effect of treatment using such compositions on amyloid plaque burden.

Anti-Amyloid Peptides: Antibodies, Analogs and Fragments of Amyloid Proteins

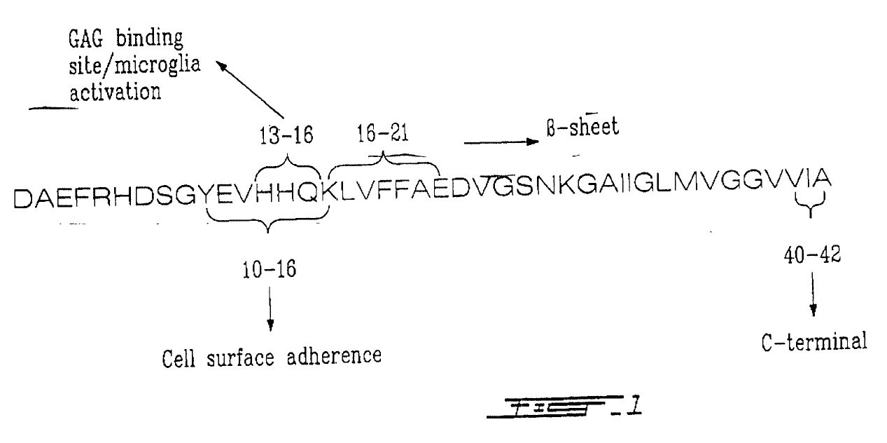

Generally, anti-amyloid peptides of the invention are composed of a specific plaque component, preferably a fibril forming component, which is usually a characteristic protein, peptide, or fragment thereof. For instance, β-amyloid peptide can be used in any of its naturally occurring forms. The human forms of Aβ are referred to as Aβ39, Aβ40, Aβ41, Aβ42 and Aβ43. The sequences of these peptides and their relationship to the APP precursor are illustrated by FIG. 1 of Hardy et al., TINS 20, 155-158 (1997). For example, Aβ42 has the sequence:

| | |
|---|---|
| H2N-Asp-Ala-Glu-Phe-Arg-His-Asp-Ser-Gly-Tyr-Glu-Val-His-His-Gln- | (SEQ ID NO:1) |
| Lys-Leu-Val-Phe-Phe-Ala-Glu-Asp-Val-Gly-Ser-Asn-Lys-Gly-Ala-Ile-Ile | |
| Gly-Leu-Met-Val-Gly-Gly-Val-Val-Ile-Ala-OH | |

Aβ41, Aβ40 and Aβ39 differ from Aβ42 by the omission of Ala, Ala-Ile and Ala-Ile-Val respectively from the C-terminal end. Aβ43 differs from Aβ42 by the presence of a threonine residue at the C-terminus.

Immunogenic fragments of Aβ are advantageous relative to the intact molecule in the present methods for several reasons. First, because only certain epitopes within Aβ induce a useful immunogenic response for treatment of Alzheimer's disease, an equal dosage of mass of a fragment containing such epitopes provides a greater molar concentration of the useful immunogenic epitopes than a dosage of intact Aβ. Second, certain immunogenic fragments of Aβ generate an immunogenic response against amyloid deposits without generating a significant immunogenic response against APP protein from which Aβ derives. Third, fragments of Aβ are simpler to manufacture than intact Aβ due to their shorter size. Fourth, fragments of Aβ do not aggregate in the same manner as intact Aβ, simplifying preparation of pharmaceutical compositions and administration thereof. Fifth, Aβ has the unusual property that it can fix and activate both classical and alternate complement cascades. In particular, it binds to Clq and ultimately to C3bi. This association facilitates binding to macrophages leading to activation of B cells. In addition, C3bi breaks down further and then binds to CR2 on B cells in a T cell dependent manner leading to a 10,000 increase in activation of these cells. This mechanism causes Aβ to generate an immune response in excess of that of other antigens.

Some immunogenic fragments of Aβ have a sequence of at least 2, 3, 5, 6, 10 or 20 contiguous amino acids from a natural peptide. Some immunogenic fragments have no more than 10, 9, 8, 7, 5 or 3 contiguous residues from Aβ. Preferred immunogenic fragments include residues 1-42 of Aβ. In some methods, the antibody specifically binds to an epitope within residues 15-20 of Aβ. In some methods, the antibody specifically binds to an epitope within residues 13-21 of Aβ. In some methods, the antibody specifically binds to an epitope within residues 10-21 of Aβ. In some methods, the antibody specifically binds to an epitope within residues 10-16 of Aβ. In some methods, the antibody specifically binds to an epitope within residues 25-35 of Aβ. The designation Aβ15-20 for example, indicates a fragment including residues 15-20 of Aβ and lacking other residues of Aβ. Other less preferred fragments include Aβ1-5, 1-6, 1-7, 1-10, 3-7, 1-3, and 1-4. These fragments require screening for activity in clearing or preventing amyloid deposits as described in the Examples before use. Fragments lacking at least one, and sometimes at least 5 or 10 C-terminal amino acid present in a naturally occurring forms of Aβ are used in some methods. For example, a fragment lacking 5 amino acids from the C-terminal end of Aβ43 includes the first 38 amino acids from the N-terminal end of Aβ. Other components of amyloid plaques, for example, synuclein, and epitopic fragments thereof can also be used to induce an immunogenic response.

Unless otherwise indicated, reference to Aβ includes the natural human amino acid sequences indicated above as well as analogs including allelic, species and induced variants. Analogs typically differ from naturally occurring peptides at one, two or a few positions, often by virtue of conservative substitutions. Analogs typically exhibit at least 50% sequence identity with natural peptides, and preferably 60%, 70%, 80% and most preferably 90% sequence identity. Some analogs also include unnatural amino acids or modifications of N or C terminal amino acids at a one, two or a few positions. For example, the natural aspartic acid residue at position 1 and/or 7 of Aβ can be replaced with iso-aspartic acid. Examples of unnatural amino acids are D-amino acids, α,α-disubstituted amino acids, N-alkyl amino acids, lactic acid, 4-hydroxyproline, y-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and isoaspartic acid. Fragments and analogs can be screened for prophylactic or therapeutic efficacy in transgenic animal models in comparison with untreated or placebo controls as described below.

Aβ, its fragments, and analogs can be synthesized by solid phase peptide synthesis or recombinant expression, or can be obtained from natural sources. Automatic peptide synthesizers are commercially available from numerous suppliers, such as Applied Biosystems, Foster City, Calif. Recombinant expression can be in bacteria, such as E. coli, yeast, insect cells or mammalian cells. Procedures for recombinant expression are described by Sambrook et al., Molecular Cloning: A Laboratory Manual (C.S.H.P. Press, NY 2d ed., 1989). Some forms of Aβ peptide are also available commercially (e.g., American Peptides Company, Inc., Sunnyvale, Calif. and California Peptide Research, Inc. Napa, Calif.).

Therapeutic peptides also include longer polypeptides that include, for example, an active fragment of Aβ peptide, together with other amino acids. Other amino acids can include those having adjuvant properties and those which serve to increase the stability of the peptide. For example, preferred peptides include fusion proteins comprising a segment of Aβ fused to a heterologous amino acid sequence that induces a helper T-cell response against the heterologous amino acid sequence and thereby a B-cell response against the Aβ segment. Such polypeptides can be screened for prophylactic or therapeutic efficacy in animal models in comparison with untreated or placebo controls as described below. The Aβ peptide, analog, active fragment or other polypeptide can be administered in associated or multimeric form or in dissociated form Therapeutic peptides also include multimers of monomeric and oligomeric immunogenic peptides. More generally, therapeutic peptides for use in the present invention produce or induce an immune response against a plaque, or more specifically, a fibril component thereof. Such peptides therefore include, but are not limited to, the component itself and variants thereof, analogs and mimetics of the component that induce and/or cross-react with antibodies to the component, as well as antibodies or T-cells that are specifically reactive with the fibril and/or amyloid peptide. According to an important feature, pharmaceutical compositions are not selected from non-specific components—that is, from those components that are generally circulating or that are ubiquitous throughout the body. By way of example, Serum Amyloid Protein (SAP) is a circulating plasma glycoprotein that is produced in the liver and binds to most known forms of amyloid deposits. Therapeutic compositions are preferably directed to this component.

Induction of an immune response can be active, as when an immunogen is administered to induce antibodies or T-cells reactive with the component in a patient, or passive, as when an antibody is administered that itself binds to the fibril and/or amyloid peptide in the patient. Exemplary peptides for inducing or producing an immune response against amyloid plaques are described below.

One general class of preferred anti-amyloid peptides consists of peptides that are derived from amyloid fibril proteins. As mentioned above, the hallmark of amyloid diseases is the deposition in an organ or organ of amyloid plaques consisting mainly of fibrils, which, in turn, are composed of characteristic fibril proteins or peptides. According to the present invention, such a fibril protein or peptide component is a useful peptide for inducing an anti-amyloid immune response. Table 1 summarizes exemplary fibril-forming proteins that are characteristic of various amyloid diseases. In accordance with this aspect of the present invention, administration to an afflicted or susceptible individual of an immunostimulatory composition which includes the appropriate fibril protein or peptide, including homologs or fragments thereof, provides therapeutic or prophylaxis with respect to the amyloid disease.

Other formulations for treating hereditary forms of amyloidosis, discussed above, include compositions that produce immune responses against gelsolin fragments for treatment of hereditary systemic amyloidosis, mutant lysozyme protein (Alys), for treatment of a hereditary neuropathy, mutant alpha chain of fibrinogen (AfibA) for a non-neuropathic form of amyloidosis manifest as renal disease, mutant cystatin C (Acys) for treatment of a form of hereditary cerebral angiopathy reported in Iceland. In addition, certain hereditary forms of prion disease (e.g., Creutzfeldt-Jacob disease (CJD), Gerstmann-Sträussler-Scheinker syndrome (GSS), and fatal familial insomnia (FFI)) are characterized by a mutant isoform of prion protein, PrP^{Sc}. This protein can be used in therapeutic compositions for treatment and prevention of deposition of PrP plaques, in accordance with the present invention.

As discussed above, amyloid deposition, either systemic or focal, is also associated with aging. It is a further aspect of the present invention that such deposition can be prevented or treated by administering to susceptible individuals compositions consisting of one or more proteins associated with such aging. Thus, plaques composed of ATTR derived from wild type TTR are frequently found in heart tissue of the elderly. Similarly, certain elderly individuals may develop asymptomatic fibrillar focal deposits of Aβ in their brains; Aβ peptide treatment, as detailed herein may be warranted in such individuals. β₂ microglobulin is a frequent component of corpora amylacea of the prostrate, and is therefore a further candidate peptide in accordance with the present invention.

By way of further example, but not limitation, there are a number of additional, non-hereditary forms amyloid disease that are candidates for treatment methods of the present invention. β₂ microglobulin fibrillar plaques commonly develop in patients receiving long term hemodialysis or peritoneal dialysis. Such patients may be treated with therapeutic compositions directed to β₂ microglobulin or, more preferably, immunogenic epitopes thereof, in accordance with the present invention.

Hormone-secreting tumors may also contain hormone-derived amyloid plaques, the composition of which are generally characteristic of the particular endocrine organ affected. Thus such fibrils may be made up of polypeptide hormones such as calcitonin (medullary carcinoma of the thyroid), islet amyloid polypeptide (occurring in most patients with Type II diabetes), and atrial natriuretic peptide (isolated atrial amyloidosis). Compositions directed at amyloid deposits which form in the aortic intima in atherosclerosis are also contemplated by the present invention. For example, Westermark, et al. describe a 69 amino acid N-terminal fragment of Apolipoprotein A which forms such plaques (Westermark, et al. Am. J. Path. 147: 1186-92, 1995); therapeutic compositions of the present invention include immunological peptides directed to such a fragment, as well as the fragment itself.

The foregoing discussion has focused on amyloid fibril components that may be used as therapeutic peptides in treating or preventing various forms of amyloid disease.

The therapeutic peptide can also be an active fragment or analog of a naturally occurring or mutant fibril peptide or protein that contains an epitope that induces a similar protective or therapeutic immune response on administration to a human. Immunogenic fragments typically have a sequence of at least 3, 5, 6, 10 or 20 contiguous amino acids from a natural peptide. Exemplary Aβ peptide immunogenic fragments include Aβ residues 15-20; residues 13-21; residues 10-21; residues 10-16; and residues 25-35 of Aβ. In some methods, the antibody binds to an epitope comprising a free N-terminal residue of Aβ.

Fragments lacking at least one, and sometimes at least 5 or 10 C-terminal amino acid present in a naturally occurring forms of the fibril component are used in some methods. For example, a fragment lacking 5 amino acids from the C-terminal end of Aβ43 includes the first 38 amino acids from the N-terminal end of AB. Fragments from the N-terminal half of Aβ are preferred in some methods. Analogs include allelic, species and induced variants. Analogs typically differ from naturally occurring peptides at one or a few positions, often by virtue of conservative substitutions. Analogs typically exhibit at least 80 or 90% sequence identity with natural peptides. Some analogs also include unnatural amino acids or modifications of N or C terminal amino acids. Examples of unnatural amino acids are alpha, alpha-disubstituted amino acids, N-alkyl amino acids, lactic acid, 4-hydroxyproline, (-carboxyglutamate, (-N,N,N-trimethyllysine, (-N-acetyllysine, O-phosphoserine, N-acetylserine, N-formylmethionine, 3-methylhistidine, 5-hydroxylysine, T-N-methylarginine.

Generally, persons skilled in the art will appreciate that fragments and analogs designed in accordance with this aspect of the invention can be screened for cross-reactivity with the naturally occurring fibril components and/or prophylactic or therapeutic efficacy in transgenic animal models as described below. Such fragments or analogs may be used in therapeutic compositions of the present invention, if their immunoreactivity and animal model efficacy is roughly equivalent to or greater than the corresponding parameters measured for the amyloid fibril components.

Such peptides, proteins, or fragments, analogs and other amyloidogenic peptides can be synthesized by solid phase peptide synthesis or recombinant expression, according to standard methods well known in the art, or can be obtained from natural sources. Exemplary fibril compositions, methods of extraction of fibrils, sequences of fibril peptide or protein components are provided by many of the references cited in conjunction with the descriptions of the specific fibril components provided herein. Additionally, other compositions, methods of extracting and determining sequences are known in the art available to persons desiring to make and use such compositions. Automatic peptide synthesizers may be used to make such compositions and are commercially available from numerous manufacturers, such as Applied Biosystems (Perkin Elmer; Foster City, Calif.), and procedures for preparing synthetic peptides are known in the art. Recombinant expression can be in bacteria, such as E. coli, yeast, insect cells or mammalian cells; alternatively, proteins can be produced using cell free in vitro translation systems known in the art. Procedures for recombinant expression are described by Sambrook et al., Molecular Cloning: A Laboratory Manual (C.S.H.P. Press, NY 2d ed., 1989). Certain peptides and proteins are also available commercially; for example, some forms of Aβ peptide are available from suppliers such as American Peptides Company, Inc., Sunnyvale, Calif., and California Peptide Research, Inc. Napa, Calif.

Therapeutic peptides may also be composed of longer polypeptides that include, for example, the active peptide fibril fragment or analog, together with other amino acids. For example, Aβ peptide can be present as intact APP protein or a segment thereof, such as the C-100 fragment that begins at the N-terminus of Aβ and continues to the end of APP. Such polypeptides can be screened for prophylactic or therapeutic efficacy in animal models as described below. The Aβ peptide, analog, active fragment or other polypeptide can be administered in associated form (i.e., as an amyloid peptide) or in dissociated form. Therapeutic peptides may also include multimers of monomeric and oligomeric immunogenic peptides or conjugates or carrier proteins, and/or, as mentioned above, may be added to other fibril components, in order to provide a broader range of anti-amyloid plaque activity.

In a further variation, an immunogenic peptide, such as a fragment of Aβ, can be presented by a virus or a bacteria as part of an immunogenic composition. A nucleic acid encoding the immunogenic peptide is incorporated into a genome or episome of the virus or bacteria. Optionally, the nucleic acid is incorporated in such a manner that the immunogenic peptide is expressed as a secreted protein or as a fusion protein with an outer surface protein of a virus or a transmembrane protein of a bacteria so that the peptide is displayed. Viruses or bacteria used in such methods should be nonpathogenic or attenuated. Suitable viruses include adenovirus, HSV, Venezuelan equine encephalitis virus and other alpha viruses, vesicular stomatitis virus, and other rhabdo viruses, vaccinia and fowl pox. Suitable bacteria include Salmonella and Shigella. Fusion of an immunogenic peptide to HBsAg of HBV is particularly suitable. Therapeutic peptides also include peptides and other compounds that do not necessarily have a significant amino acid sequence similarity with Aβ but nevertheless serve as mimetics of Aβ and induce a similar immune response. For example, any peptides and proteins forming β-pleated sheets can be screened for suitability. Anti-idiotypic antibodies against monoclonal antibodies to Aβ or other amyloidogenic peptides can also be used. Such anti-Id antibodies mimic the antigen and generate an immune response to it (see Essential Immunology (Roit ed., Blackwell Scientific Publications, Palo Alto, 6th ed.), p. 181). Peptides other than Aβ peptides should induce an immunogenic response against one or more of the preferred segments of Aβ listed above (e.g., 10-16, 10-21, 13-21, and 25-35). Preferably, such peptides induce an immunogenic response that is specifically directed to one of these segments without being directed to other segments of Aβ.

Random libraries of peptides or other compounds can also be screened for suitability. Combinatorial libraries can be produced for many types of compounds that can be synthesized in a step-by-step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated by reference for all purposes). Peptide libraries can also be generated by phage display methods. See, e.g., Devlin, WO 91/1 8980.

Combinatorial libraries and other compounds are initially screened for suitability by determining their capacity to bind to antibodies or lymphocytes (B or T) known to be specific for Aβ or other amyloidogenic peptides such as ATTR. For example, initial screens can be performed with any polyclonal sera or monoclonal antibody to Aβ or any other amyloidogenic peptide of interest. Compounds identified by such screens are then further analyzed for capacity to induce antibodies or reactive lymphocytes to Aβ or other amyloidogenic peptide. For example, multiple dilutions of sera can be tested on microtiter plates that have been precoated with fibril peptide, and a standard ELISA can be performed to test for reactive antibodies to Aβ. Compounds can then be tested for prophylactic and therapeutic efficacy in transgenic animals predisposed to an amyloidogenic disease, as described in the Examples. Such animals include, for example, mice bearing a 717 mutation of APP described by Games et al., supra, and mice bearing a 670/671 Swedish mutation of APP such as described by McConlogue et al., U.S. Pat. No. 5,612,486 and Hsiao et al., Science 274, 99 (1996); Staufenbiel et al., Proc. Natl. Acad. Sci. USA 94, 13287-13292 (1997); Sturchler-Pierrat et al., Proc. Natl. Acad. Sci. USA 94, 13287-13292 (1997); Borchelt et al., Neuron 19, 939-945 (1997)). The same screening approach can be used on other potential peptides such as fragments of Aβ, analogs of Aβ and longer peptides including Aβ, described above.

It is appreciated that immunological responses directed at other amyloid plaque components can also be effective in preventing, retarding or reducing plaque deposition in amyloid diseases. Such components may be minor components of fibrils or associated with fibrils or fibril formation in the plaques, with the caveat that components that are ubiquitous throughout the body, or relatively non-specific to the amyloid deposit, are generally less suitable for use as therapeutic targets.

It is therefore a further discovery of the present invention that peptides that induce an immune response to specific plaque components are useful in treating or preventing progression of amyloid diseases. This section provides background on several exemplary amyloid plaque-associated molecules. Induction of an immune response against any of these molecules, alone or in combination with immunogenic therapeutic compositions against the fibril components described above or against any of the other non-fibril forming components described below, provides an additional anti-amyloid treatment regimen, in accordance with the present invention. Also forming part of the present invention are passive immunization regimens based on such plaque components, as described herein.

By way of example, synuclein is a protein that is structurally similar to apolipoproteins but is found in neuronal cytosol, particularly in the vicinity of presynaptic terminals. There are at least three forms of the protein, termed alpha, beta and gamma synuclein. Recently, it has been shown that alpha and β synuclein are involved in nucleation of amyloid deposits in certain amyloid diseases, particularly Alzheimer's disease. (Clayton, D. F., et al., TINS 21(6): 249-255, 1998). More specifically, a fragment of the NAC domain of alpha and β synuclein (residues 61-95) has been isolated from amyloid plaques in Alzheimer's patients; in fact this fragment comprises about 10% of the plaque that remains insoluble after solubilization with sodium dodecyl sulfate (SDS). (George, J. M., et al. Neurosci. News 1: 12-17, 1995). Further, both the full length alpha synuclein and the NAC fragment thereof have been reported to accelerate the aggregation of β-amyloid peptide into insoluble amyloid in vitro. (Clayton, supra).

Additional components associated with amyloid plaques include non-peptide components. For example, perlecan and perlecan-derived glycosaminoglycans are large heparin sulfate proteoglycans that are present in Aβ-containing amyloid plaques of Alzheimer's disease and other CNS and systemic amyloidoses, including amylin plaques associated with diabetes. These compounds have been shown to enhance Aβ fibril formation. Both the core protein and glycosaminoglycan chains of perlecan have been shown to participate in binding to Aβ. Additional glycosaminoglycans, specifically, dermatan sulfate, chondroitin-4-sulfate, and pentosan polysulfate, are commonly found in amyloid plaques of various types and have also been shown to enhance fibril formation. Dextran sulfate also has this property. This enhancement is significantly reduced when the molecules are de-sulfated. Immunogenic therapeutics directed against the sulfated forms of glycosaminoglycans, including the specific glycosaminoglycans themselves, form an additional embodiment of the present invention, either as a primary or secondary treatment. Production of such molecules, as well as appropriate therapeutic compositions containing such molecules, is within the skill of the ordinary practitioner in the art.

Immunization Procedures

The elicited antibodies present in the host having received the vaccine of the present invention bind at the Aβ(6-21) site or other sites such as Aβ(10-21), Aβ(13-21) and the C-terminal region of Aβ and have the ability to prevent amyloidogenesis. The vaccine of the present invention causes the generation of effective antiamyloidogenic antibodies in the vaccinated host.

A suggested immunization procedure is as follows:

a) prepare a vaccine from an all-D peptide having a sequence substantially the same as that of a naturally occurring β amyloid peptide, namely Aβ(all-L). The all-D peptide includes a full length Aβ(1-42, all-D), a peptide derived from an immunogenic fragment of Aβ(1-42, all-D), and a related peptidomimetic;

b) immunize a host with the vaccine to generate an antibody in the host with a binding site capable of preventing fibrillogenesis, associated cellular toxicity and neurodegeneration.

Suitable pharmaceutically acceptable carriers include, without limitation, any non-immunogenic pharmaceutical adjuvants suitable for oral, parenteral, intravascular (IV), intraarterial (IA), intramuscular (IM), and subcutaneous (SC) administration routes, such as phosphate buffer saline (PBS).

The pharmaceutical carriers may contain a vehicle, which carries antigens to antigen-presenting cells. Examples of vehicles are liposomes, immune-stimulating complexes, microfluidized squalene-in-water emulsions, microspheres which may be composed of poly(lactic/glycolic) acid (PLGA). Particulates of defined dimensions (<5 micron) include, without limitation, oil-in-water microemulsion (MF59) and polymeric microparticules.

The carriers of the present invention may also include chemical and genetic adjuvants to augment immune responses or to increase the antigenicity of antigenic immunogens. These adjuvants exert their immunomodulatory properties through several mechanisms such as lymphoid cells recruitment, cytokine induction, and the facilitation of DNA entry into cells. Cytokine adjuvants include, without limitation, granulocyte-macrophage colony-stimulating factor, interleukin-12, GM-CSF, synthetic muramyl dipeptide analog or monophosphoryl lipid A. Other chemical adjuvants include, without limitation, lactic acid bacteria, Al(OH)₃, muramyl dipeptides and saponins.

The peptide may be coupled to a carrier that will modulate the half-life of the circulating peptide. This will allow the control on the period of protection. The peptide-carrier may also be emulsified in an adjuvant and administrated by usual immunization route.

The vaccine of the present invention will, for the most part, be administered parenterally, such as intravascularly (IV), intraarterially (IA), intramuscularly (IM), subcutaneously (SC), or the like. In some instances, administration may be oral, nasal, rectal, transdermal or aerosol, where the nature of the vaccine allows for transfer to the vascular system. Usually a single injection will be employed although more than one injection may be used, if desired. The vaccine may be administered by any convenient means, including syringe, trocar, catheter, or the like. Preferably, the administration will be intravascularly, where the site of introduction is not critical to this invention, preferably at a site where there is rapid blood flow, e.g., intravenously, peripheral or central vein. Other routes may find use where the administration is coupled with slow release techniques or a protective matrix.

The use of the vaccine of the present invention in preventing and/or treating Alzheimer's disease and other amyloid related diseases can be validated by raising antibodies against the corresponding all-D peptide and testing them to see if they can effectively inhibit or prevent the fibrillogenesis of the natural amyloid peptide (all-L).

The compounds used to prepare vaccines in accordance with the present invention have the common structure of Formula I:

R′—(P)—R″ (I),

wherein

P is an all-D peptide of a fibril or amyloid protein, e.g., β sheet region, GAG-binding site region, Aβ(1-42, all-D), and macrophage adherence region (10-16, all-D)immunogenic fragments thereof, immunogenic derivatives thereof, protein conjugates thereof, immunogenic peptides thereof, and immunogenic peptidomimetics thereof;

R′ is an N-terminal substituent selected from the group consisting of:

hydrogen;

lower alkyl groups, e.g., acyclic or cyclic having 1 to 8 carbon atoms, without or with functional groups, e.g., carboxylate, sulfonate and phosphonate;

aromatic groups;

heterocyclic groups; and

acyl groups, e.g., alkylcarbonyl, arylcarbonyl, sulfonyl and phosphonyl groups; and

R″ is a C-terminal substituent, e.g., hydroxy, alkoxy, aryloxy, unsubstituted or substituted amino groups.

R′ and R″ may be identical or different; the alkyl or aryl group of R′ and R″ may further be substituted with organic functionalities selected from the group of halides (F, Cl, Br, and I), hydroxyl, alkoxyl, aryloxyl, hydroxycarbonyl, alkoxylcarbonyl, aryloxycarbonyl, carbamyl, unsubstituted or substituted amino, sulfo or alkyloxysulfonyl, phosphono or alkoxyphosphonyl, and the like.

Where a functional group is an acid, its pharmaceutically acceptable salt or ester is in the scope of this invention. Where a functional group is a base, its pharmaceutically acceptable salt is in the scope of this invention.

In one embodiment, P is a peptide capable of interacting with at least one region of a fibril or amyloid protein.

In another embodiment, the preferred compounds are selected from the full-length peptide, Aβ(1-42, all-D), and its lower homologues consisting of Aβ(1-40, all-D), Aβ(1-35, all-D), Aβ(1-28, all-D), and Aβ(10-21, all-D).

In another embodiment, the preferred compounds are selected from a group of short peptides, e.g., Aβ(1-7, all-D), Aβ(10-16, all-D), Aβ(16-21, all-D), Aβ(36-42, all-D). The peptides can be shortened further by removing one or more residues from either end or both ends.

The preferred compounds may also be all-D peptides derived from the peptides above by substitution of one or more residues in the naturally occurring sequence. In another embodiment, the preferred compounds are peptidomimetics of the above-said peptides.

In a further embodiment, the preferred compounds may be coupled with a carrier that will modulate the biodistribution, immunogenic property and the half-life of the compounds.

The following are exemplary compounds for preparing vaccines for preventing or treating Alzheimer's disease and other amyloid related diseases:

| | |
|---|---|
| SEQ ID NO: 1 | Aβ(1-42, all-D) |
| | DAEFRHDSGYEVHHQKLVFFAEDVGSNKGAIIGLMVGGVVIA |
| SEQ ID NO: 2 | Aβ(1-40, all-D) |
| | DAEFRHDSGYEVHHQKLVFFAEDVGSNKGAIIGLMVGGVV |
| SEQ ID NO: 3 | Aβ(1-35, all-D) |
| | DAEFRHDSGYEVHHQKLVFFAEDVGSNKGAIIGLM |
| SEQ ID NO: 4 | Aβ(1-28, all-D) |
| | DAEFRHDSGYEVHHQKLVFFAEDVGSNK |
| SEQ ID NO: 5 | Aβ(1-7, all-D) |
| | DAEFRHD |
| SEQ TD NO: 6 | Aβ(10-16, all-D) |
| | YEVHHQK |
| SEQ ID NO: 7 | Aβ(16-21, all-D) |
| | KLVFFA |
| SEQ ID NO: 8 | Aβ(10-21, all-D) |
| | YEVHHQKLVFFA |
| SEQ ID NO: 9 | Aβ(13-21, all-D) |
| | HHQKLXTFA |
| SEQ ID NO: 10 | Aβ(36-42, all-D) |
| | VGGVVIA |
| SEQ ID NO: 11 | Lys-Ile-Val-Phe-Phe-Ala (all-D) |
| SEQ TD NO: 12 | Lys-Lys-Leu-Val-Phe-Phe-Ala (all-D) |
| SEQ ID NO: 13 | Lys-Phe-Val-Phe-Phe-Ala (all-D) |
| SEQ ID NO: 14 | Ala-Phe-Phe-Val-Leu-Lys (all-D) |
| SEQ ID NO: 15 | Lys-Leu-Val-Phe (all-D) |
| SEQ ID NO: 16 | Lys-Ala-Val-Phe-Phe-Ala (all-D) |
| SEQ ID NO: 17 | Lys-Leu-Val-Phe-Phe (all-D) |
| SEQ ID NO: 18 | Lys-Val-Val-Phe-Phe-Ala (all-D) |
| SEQ ID NO: 19 | Lys-Ile-Val-Phe-Phe-Ala-NH₂ (all-D) |
| SEQ ID NO: 20 | Lys-Leu-Val-Phe-Phe-Ala-NH₂ (all-D) |
| SEQ ID NO: 21 | Lys-Phe-Val-Phe-Phe-Ala-NH₂ (all-D) |
| SEQ ID NO: 22 | Ala-Phe-Phe-Val-Leu-Lys-NH₂ (all-D) |
| SEQ ID NO: 23 | Lys-Leu-Val-Phe-NH₂ (all-D) |
| SEQ ID NO: 24 | Lys-Ala-Val-Phe-Phe-Ala-NH₂ (all-D) |
| SEQ ID NO: 25 | Lys-Leu-Val-Phe-Phe-NH₂ (all-D) |
| SEQ ID NO: 26 | Lys-Val-Val-Phe-Phe-Ala-NH₂ (all-D) |
| SEQ ID NO: 27 | Lys-Leu-Val-Phe-Phe-Ala-Gln (all-D) |
| SEQ ID NO: 28 | Lys-Leu-Val-Phe-Phe-Ala-Gln-NH₂ (all-.D) |
| SEQ ID NO: 29 | His-His-Gln-Lys-Leu-Val-Phe-Phe-Ala-Gln (all-D) |
| SEQ ID NO: 30 | Asp-Asp-Asp (all-D) |
| SEQ ID NO: 31 | Lys-Val-Asp-Asp-Gln-Asp (all-D) |
| SEQ ID NO: 32 | His-His-Gln-Lys (all-D) |
| SEQ ID NO: 33 | Phe-Phe-NH-CH₂CH₂SO₃H (all-D) |
| SEQ ID NO: 34 | Phe-Phe-NH-CH₂CH₂CH₂SO₃H (all-D) |
| SEQ ID NO: 35 | Phe-Phe-NH-CH₂CH₂CH₂CH₂SO₃H (all-D) |
| SEQ ID NO: 36 | Phe-Tyr-NH-CH₂CH₂SO₃H (all-D) |
| SEQ ID NO: 37 | Phe-Tyr-NH-CH₂CH₂CH₂SO₃H (all-D) |
| SEQ ID NO: 38 | Phe-Tyr-NH-CH₂CH₂CH₂CH₂SO₃H (all-D) |
| SEQ ID NO: 39 | HO₃SCH₂CH₂-Phe-Phe (all-D) |
| SEQ ID NO: 40 | HO₃SCH₂CH₂CH₂-Phe-Phe (all-D) |
| SEQ ID NO: 41 | HO₃SCH₂CH₂CH₂-Phe-Phe (all-D) |
| SEQ ID NO: 42 | HO₃SCH₂CH₂CH₂-Phe-Tyr (all-D) |
| SEQ ID NO: 43 | HO₃SCH₂CH₂CH₂-Phe-Tyr (all-D) |
| SEQ ID NO: 44 | HO₃SCH₂CH₂CH₂-Phe-Tyr (all-D) |
| SEQ ID NO: 45 | HO₃SCH₂CH₂CH₂-Leu-Val-Phe-Phe-Ala (all-D) |
| SEQ ID NO: 46 | HO₃SCH₂CH₂CH₂-Leu-Val-Phe-Phe-Ala (all-D) |
| SEQ ID NO: 47 | HO₃SCH₂CH₂CH₂-Leu-Val-Phe-Phe-Ala (all-D) |
| SEQ ID NO: 48 | Leu-Val-Phe-Phe-Ala-NH-CH₂CH₂SO₃H (all-D) |
| SEQ ID NO: 49 | Leu-Val-Phe-Phe-Ala-NH-CH₂CH₂CH₂SO₃H (all-D) |
| SEQ ID NO: 50 | Leu-Val-Phe-Phe-Ala-NH-CH₂CH₂CH₂CH₂SO₃H (all-D). |

The compounds listed above may be modified by removing or inserting one or more amino acid residues, or by substituting one or more amino acid residues with other amino acid or non-amino acid fragments.

The following are exemplary compounds derived from compound 18 (all-D KLVFFA-NH₂; SEQ ID NO: 18) by substituting one or two amino acid residue(s) with other amino acids.

| | |
|---|---|
| SEQ ID NO: 51 | Lys-Leu-Val-Trp-Phe-Ala-NH₂(all-D) |
| SEQ ID NO: 52 | Lys-Leu-Val-Phe-Trp-Ala- NH₂(all-D) |
| SEQ ID NO: 53 | Lys-Leu-Val-Trp-Trp-Ala- NH₂(all-D) |
| SEQ ID NO: 54 | Lys-Leu-Val-Tyr-Phe-Ala- NH₂(all-D) |
| SEQ ID NO: 55 | Lys-Leu-Val-Phe-Tyr-Ala- NH₂(all-D) |
| SEQ ID NO: 56 | Lys-Leu-Val-Tyr-Tyr-Ala- NH₂(all-D) |
| SEQ ID NO: 57 | Lys-Leu-Val-Thi-Phe-Ala- NH₂ (all-D) |
| SEQ ID NO: 58 | Lys-Leu-Val-Phe-Thi-Ala- NH₂(all-D) |
| SEQ ID NO: 59 | Lys-Leu-Val-Thi-Thi-Ala- NH₂(all.-D) |
| SEQ ID NO: 60 | Lys-Leu-Val-Cha-Phe-Ala- NH₂(all-D) |
| SEQ ID NO: 61 | Lys-Leu-Val-Phe-Cha-Ala- NH₂(all-D) |
| SEQ ID NO: 62 | Lys-Leu-Val-Cha-Cha-Ala- NH₂(all-D) |
| SEQ ID NO: 63 | Lys-Leu-Val-Pgly-Phe-Ala- NH₂(all-D) |
| SEQ ID NO: 64 | Lys-Leu-Val-Phe-Pgly-Ala- NH₂(all-D) |
| SEQ ID NO: 65 | Lys-Leu-Val-Pgly-Pgly-Ala- NH₂(all-D). |

For the above compounds, the terms Thi, Cha and Pgly are intended to mean thienylalanine, cyclohexylalanine and phenylglycine, respectively.

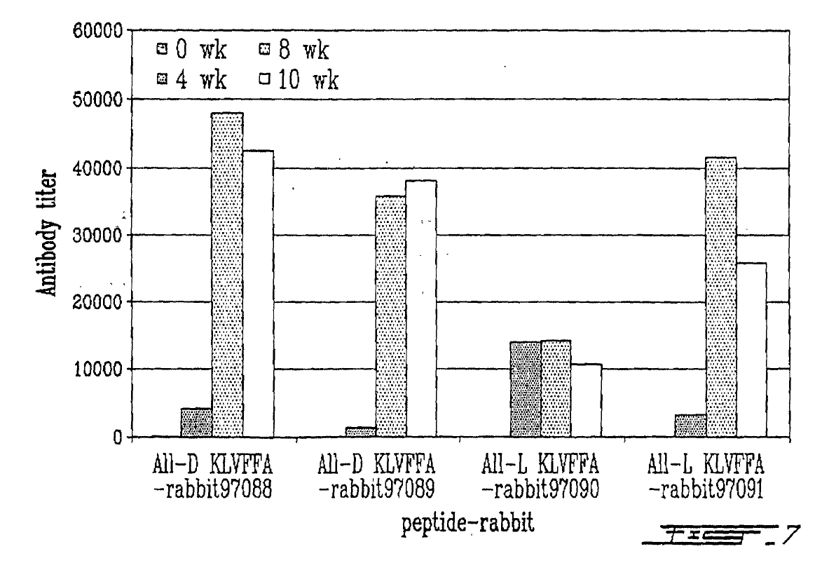

Rabbits were immunized with all-D or all-L KLVFFA. Results of the antibody titers obtained are shown in FIG. 7. As seen in FIG. 7, the vaccine of the present invention causes production of antibodies.

The present invention encompasses various types of immune responses triggered using the vaccine of the present invention, e.g., amyloid therapies using the vaccine approach.

In accordance with the present invention, there is also provided a vaccine which triggers a preferential TH-2 response or a TH-1 response, according to the type of immunization used. By inducing a TH-2 response, anti-inflammatory cytokine production such as IL-4, I1-10 and TGF-β, as well as the production of IgG 1 and IgG 2b antibody classes, are favored. Such type of response would be preferred, as a major inflammatory response in the brain of the patients with AD would be avoided. On the other hand, with a preferred TH-1 response, a pro-inflammatory response with a production of inflammatory cytokines such as IL-1, Il-6, TNF and IFN gamma would be favored. This type of response would more likely trigger activation of the macrophage population. These macrophages would then phagocytose any particulate deposits (such as plaques) via a complement-activated process as well as via antibody-mediated process. This approach would be beneficial to clear already organized senile plaques and prevent the formation of new fibrillary deposits.

Both approaches (i.e. TH-1 and TH-2) are of value. The antigen used could be the peptides which contain regions responsible for cellular adherence, i.e., region 10-16, regions responsible for the GAG binding site, i.e., 13-16, regions responsible for the β sheet 16-21 or regions for 40-42. These peptides could be presented in such a way that either a preferential TH-1 or TH-2 response is obtained, depending on the type of adjuvant used, or depending on the route of administration of the vaccine. For example, a mucosal immunization via nasal administration is possible, since it is known that such a route of administration would favor a TH-2 response.

The present invention will be more readily understood by referring to the following examples, which are given to illustrate the invention rather than to limit its scope.

### EXAMPLE I

An in vitro validation procedure to test the effectiveness of all-D peptide vaccines derived from fibrillogenic proteins was performed in rabbits or mice to demonstrate that antibodies can be raised against Aβ 16-21 (all-D) (see FIG. 7). The antibodies produced were tested to prove that they effectively prevent the fibrillogenesis of natural Aβ(1-40, all-L) in vitro. Standard assays for fibrillogenesis were used to evaluate activity, such as those based on Thioflavine T, circular dichroism and solubility.

This approach could also be used to establish which areas of the Aβ peptide are most effective when used in the form of all-D peptides to prepare antifibrillogenic vaccines. One way this could be performed is as follows:

a) rabbits or mice are immunized with a series of overlapping all-D peptides generated from the Aβ(1-42) sequence, e.g., Aβ(1-6), Aβ(2-8), Aβ(4-10), etc.

b) antisera are prepared from the immunized rabbits or mice.

c) these antisera are tested to see which parts of the Aβ sequence produce antisera which most effectively prevents fibrillogenesis in the standard assays for fibrillogenesis mentioned above.

### EXAMPLE II

Effect of Antibodies Against D- and L-Aβ(16-21) Peptide Vaccine on Fibrillogenesis

A validation procedure to test anti-fibrillogenic activity of antibodies raised against D-and L-Aβ(16-21) peptide was performed.

Rabbits were immunized with D- or L-Aβ(16-21) peptide. Antibodies raised were tested for their antifibrillogenic activities by ThT assay and by electron microscopy (EM).

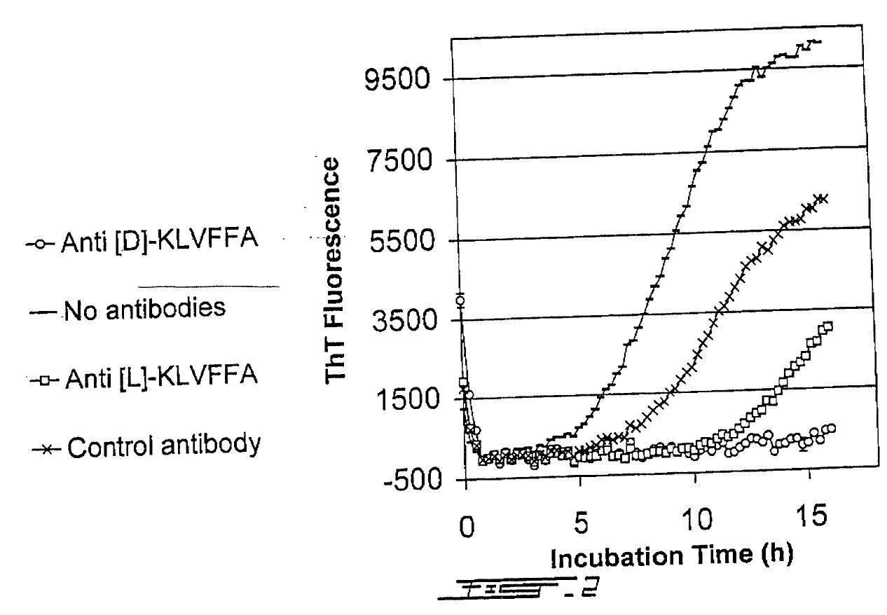

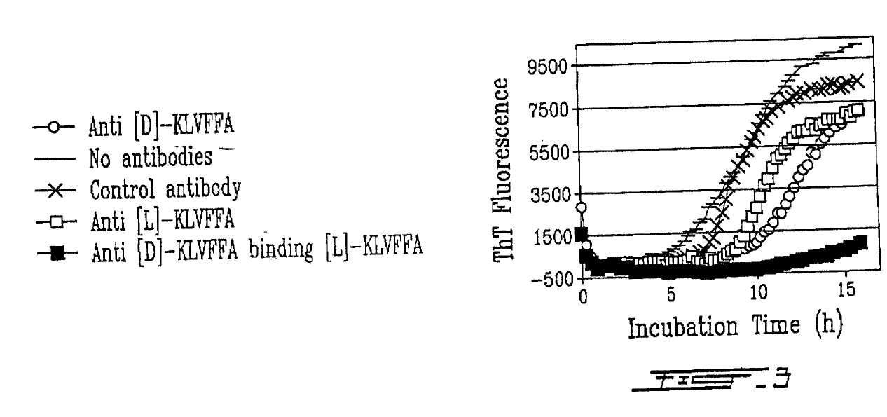

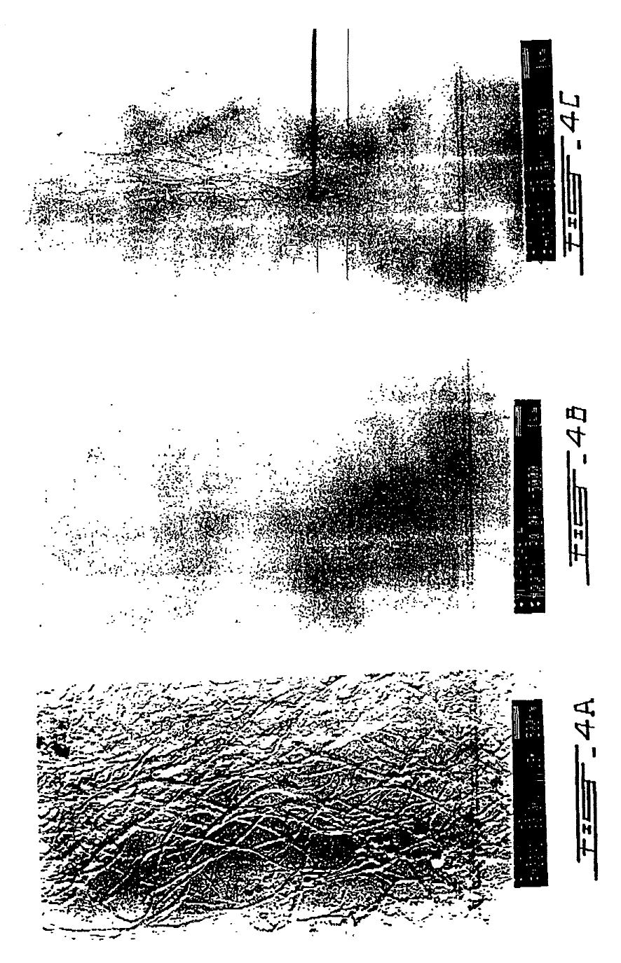

Antibodies raised against the D- and L-forms of KLVFFA were capable of blocking the fibrillogenesis process as seen either by the Thioflavin T assay (ThT) (FIGS. 2 and 3) and by EM (FIGS. 4A to 4C). In the ThT assay, fibril formation is monitored by the increase in fluorescence with time. As seen in the Figures, the antibodies were capable of inhibiting such an increase in fluorescence, proving that these antibodies were inhibiting fibrillogenesis.

As can be seen in these figures (FIGS. 2 to 4), antibodies raised against the D-peptide have a better anti-fibrillogenic activity than anti-L antibodies.

These results were also confirmed by EM (FIGS. 4A to 4C) where both anti-D and anti-L KLVFFA peptide blocked the fibril formation when compared to control (FIG. 4A). Moreover, again the anti-D peptide has a greater anti-fibrillogenic activity (FIG. 4B) than the anti-L peptide (FIG. 4C). This goes along with the ThT assay where the decrease in fluorescence was greater with the anti-D peptide antibody than with the anti-L peptide antibody.

### EXAMPLE III

### Antibody Binding Assay

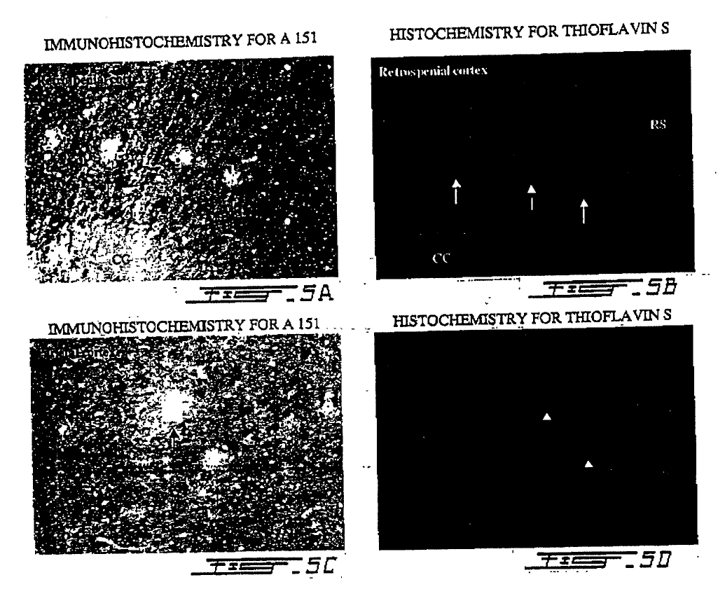

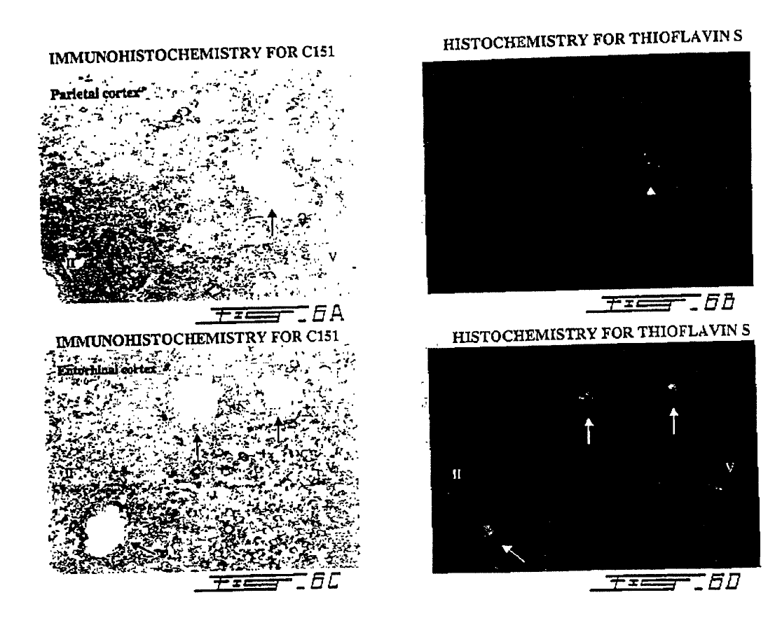

Brain sections were stained with antibodies raised against KLVFFA peptide (D and L forms). As seen in FIGS. 5A to 5D and 6A to 6D, the antibodies were not capable of binding to aggregated (ThioS positive) Aβ. It can be seen from both sets of figures, which were stained for both plaques (ThioS) and anti-peptides that the antibodies are recognizing Aβ at the surface of the cells but are not capable of binding to plaques. These results show that the anti-KLVFFA peptide antibody is recognizing the non-fibrillary Aβ but does not bind to aggregated Aβ. There was no difference between the anti-D and anti-L peptide antibodies in this assay.

These results clearly prove that the antibody recognizes only the non-aggregated form and blocks the fibrillogenesis. By having such activity, the vaccine of the present invention 1) prevents Aβ from organizing itself into a fibril and 2) prevents an inflammatory response being triggered by such an antibody binding to an insoluble form, since the antibody is not able to bind to aggregated Aβ.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for preventing or treating an amyloid-related disease in a subject, comprising: administering to the subject an antigenic amount of an all-D peptide, wherein said all-D peptide elicits the production of antibodies against said all-D peptide and induces an immune response by said subject, thereby preventing or reducing amyloid-induced cellular toxicity or amyloid fibril formation.

2. A method for preventing or treating an amyloid-related disease in a subject, comprising: administering to the subject an antigenic amount of an all-D peptide, wherein said all-D peptide elicits the production of antibodies against said all-D peptide and induces an immune response by said subject, thereby preventing or reducing amyloid-induced cellular toxicity or amyloid fibril formation.

3. The method of claim 1, wherein said all-D peptide interacts with at least one region of an amyloid protein, said region being selected from the group consisting of: C-terminal region, β sheet region, GAG-binding site region, macrophage adherence region, immunogenic fragments thereof, protein conjugates thereof, immunogenic derivative peptides thereof, immunogenic peptides thereof, and immunogenic peptidomimetics thereof.

4. The method of claim 3, wherein said all-D peptide further comprises:
(a) an N-terminal substituent selected from the group consisting of:
hydrogen;
lower alkyl group consisting of acyclic or cyclic having 1 to 8 carbon atoms;
aromatic group;
heterocyclic group; and
acyl group; and
(b) a C-terminal substituent selected from the group consisting of hydroxy, alkoxy, aryloxy, unsubstituted and substituted amino groups.

5. The method of claim 4, wherein said alkyl or aromatic group is further substituted with a group selected from the group consisting of halide, hydroxyl, alkoxyl, aryloxyl, hydroxycarbonyl, alkoxylcarbonyl, aryloxycarbonyl, carbamyl, unsubstituted amino, substituted amino, sulfo, alkyloxysulfonyl, phosphono and alkoxyphosphonyl groups.

6. The method of claim 1, wherein said all-D peptide further comprises an acid functional group, or a pharmaceutically acceptable salt or ester form thereof.

7. The method of claim 3, wherein said all-D peptide further comprises a base functional group, or a pharmaceutically acceptable salt form thereof.

8. The method of claim 4, wherein said all-D peptide is selected from the group consisting of SEQ ID NOS: 1-50.

9. The method of claim 8, wherein said all-D peptide is modified by substituting at least one amino acid residue with another amino acid or non-amino acid fragment.

10. The method of claim 9, wherein said modified peptide is selected from the group consisting of SEQ ID NOS: 49-65.

11. The method of claim 8, wherein said all-D peptide is modified by removing or inserting at least one amino acid residue.

12. A method for preventing or treating an amyloid-related disease in a subject, comprising: administering to the subject an antigenic amount of a peptide having Formula I:
R′—(P)—R″ (I)
wherein
P is an all-D peptide that interacts with at least one region of an amyloid protein selected from the group consisting of: C-terminal region, β sheet region, GAG-binding site region, macrophage adherence region, immunogenic fragments thereof, protein conjugates thereof, immunogenic derivative peptides thereof, immunogenic peptides thereof, and immunogenic peptidomimetics thereof;
R′ is an N-terminal substituent selected from the group consisting of:
hydrogen;
lower alkyl group consisting of acyclic or cyclic having 1 to 8 carbon atoms;
aromatic group;
heterocyclic group; and
acyl group; and
R″ is a C-terminal substituent selected from the group consisting of hydroxy group, alkoxy group, aryloxy group, unsubstituted group, and substituted amino group.

13. The method of claim 11, wherein said all-D peptide elicits the production of antibodies against said all-D peptide, and induces an immune response by said subject, thereby preventing or reducing amyloid-induced cellular toxicity or amyloid fibril formation.

14. The method of claim 11, wherein said alkyl or aromatic group is further substituted with a group selected from the group consisting of halide, hydroxyl, alkoxyl, aryloxyl, hydroxycarbonyl, alkoxylcarbonyl, aryloxycarbonyl, carbamyl, unsubstituted amino, substituted amino, sulfo, alkyloxysulfonyl, phosphono and alkoxyphosphonyl groups.

15. The method of claim 11, wherein said all-D peptide further comprises an acid functional group, or a pharmaceutically acceptable salt or ester form thereof.

16. The method of claim 11, wherein said all-D peptide further comprises a base functional group, or pharmaceutically acceptable salt form thereof.

17. The method of claim 11, wherein said all-D peptide is selected from the group consisting of SEQ ID NOS: 1-50.

18. The method of claim 17, wherein said all-D peptide is modified by substituting one or more amino acid residues with other amino acid or non-amino acid fragment.

19. The method of claim 18, wherein said modified peptide is selected from the group consisting of SEQ ID NOS: 51-65.

20. The method of claim 17, wherein said all-D peptide is modified by removing or inserting one or more amino acid residues.

21. A composition for preventing or treating an amyloid-related disease in a subject, comprising an antigenic amount of an all-D peptide, wherein said all-D peptide elicits the production of antibodies against said all-D peptide, and induces an immune response by said subject, thereby preventing or reducing amyloid-induced cellular toxicity or amyloid fibril formation.

22. The composition of claim 21, said all-D peptide interacts with at least one region of an amyloid protein, said region being selected from the group consisting of: C-terminal region, β sheet region, GAG-binding site region, macrophage adherence region, immunogenic fragments thereof, protein conjugates thereof, immunogenic derivative peptides thereof, immunogenic peptides thereof, and immunogenic peptidomimetics thereof.

23. The composition of claim 21, wherein said all-D peptide further comprises:
(a) an N-terminal substituent selected from the group consisting of:
hydrogen;
lower alkyl group consisting of acyclic or cyclic having b 1 to 8 carbon atoms;
aromatic group;
heterocyclic group; and
acyl group; and
(b) a C-terminal substituent selected from the group consisting of hydroxy, alkoxy, aryloxy, unsubstituted and substituted amino group.

24. The composition of claim 23, wherein said alkyl or aromatic group is further substituted with a group selected from the group consisting of halide, hydroxyl, alkoxyl, aryloxyl, hydroxycarbonyl, alkoxylcarbonyl, aryloxycarbonyl, carbamyl, unsubstituted amino, substituted amino, sulfo, alkyloxysulfonyl, phosphono and alkoxyphosphonyl groups.

25. The composition of claim 24, wherein said all-D peptide further comprises an acid functional group, or a pharmaceutically acceptable salt or ester form thereof.

26. The composition of claim 23, wherein said all-D peptide further comprises a base functional group, or a pharmaceutically acceptable salt form thereof.

27. The composition of claim 23, wherein said all-D peptide is selected from the group consisting of SEQ ID NOS: 1-50.

28. The composition of claim 27, wherein said all-D peptide is modified by substituting at least one amino acid residue with another amino acid or non-amino acid fragment.

29. The composition of claim 28, wherein said modified peptide is selected from the group consisting of SEQ ID NOS: 51-65.

30. The composition of claim 27, wherein said all-D peptide is modified by removing or inserting at least one amino acid residue.

31. A composition for preventing or treating an amyloid-related disease in a subject, comprising an antigenic amount of a peptide having Formula I:
R′—(P)—R″ (I)
wherein
P is an all-D peptide that interacts with at least one region of an amyloid protein selected from the group consisting of: C-terminal region, β sheet region, GAG-binding site region, macrophage adherence region, immunogenic fragments thereof, protein conjugates thereof, immunogenic derivative peptides thereof, immunogenic peptides thereof, and immunogenic peptidomimetics thereof;
R′ is an N-terminal substituent selected from the group consisting of:
hydrogen;
lower alkyl group consisting of acyclic or cyclic having 1 to 8 carbon atoms;
aromatic group;
heterocyclic group; and
acyl group; and
R″ is a C-terminal substituent selected from the group consisting of hydroxy group, alkoxy group, aryloxy group, unsubstituted group, and substituted amino group.

32. The composition of claim 31, wherein said all-D peptide elicits the production of antibodies against said all-D peptide, and induces an immune response by said subject, thereby preventing or reducing amyloid-induced cellular toxicity or amyloid fibril formation.

33. The composition of claim 31, wherein said alkyl or aromatic group is further substituted with a group selected from the group consisting of halide, hydroxyl, alkoxyl, aryloxyl, hydroxycarbonyl, alkoxylcarbonyl, aryloxycarbonyl, carbamyl, unsubstituted amino, substituted amino, sulfo, alkyloxysulfonyl, phosphono and alkoxyphosphonyl groups.

34. The composition of claim 31, wherein said all-D peptide further comprises an acid functional group, or a pharmaceutically acceptable salt or ester form thereof.

35. The composition of claim 31, wherein said all-D peptide further comprises a base functional group, or pharmaceutically acceptable salt form thereof.

36. The composition of claim 31, wherein said all-D peptide is selected from the group consisting of SEQ ID NOS: 1-50.

37. The composition of claim 36, wherein said all-D peptide is modified by substituting one or more amino acid residues with other amino acid or non-amino acid fragment.

38. The composition of claim 37, wherein said modified peptide is selected from the group consisting of SEQ ID NOS: 51-65.

39. The composition of claim 36, wherein said all-D peptide is modified by removing or inserting one or more amino acid residues.
